(19) United States
(12) Patent Application Publication
(10) Pub. No.: US 2024/0165419 A1
(43) Pub. Date: May 23, 2024

(54) SYSTEMS AND METHODS FOR MACHINE-LEARNING GUIDED TREATMENT PLANNING AND MONITORING OF ELECTRIC FIELD THERAPY IMPLANTS

(71) Applicant: Dignity Health, San Francisco, CA (US)

(72) Inventor: Benjamin Hendricks, San Francisco, CA (US)

(21) Appl. No.: 18/551,430

(22) PCT Filed: Apr. 4, 2022
(86) PCT No.: PCT/US2022/023301

§ 371 Date: Sep. 20, 2023

Related U.S. Application Data
(60) Provisional application No. 63/170,512, filed on Apr. 4, 2021.

(51) Int. Cl. A61N 1/40; A61B 5/0536; A61B 5/055; G06T 17/20; G06V 10/70

(52) U.S. Cl. CPC A61N 1/40; A61B 5/0536; A61B 5/055; G06T 17/20; G06V 10/70; G06T 2210/41; G06V 2201/03

(57) ABSTRACT

Electric field therapy requires the strategic delivery of electrical energy to living tissue. Machine learning in the context of a computer-implemented planning environment can be adopted to enhance planning, monitoring, and modulation of this therapy.

### CROSS-REFERENCE TO RELATED APPLICATIONS

This is a PCT application that claims benefit to U.S. Provisional patent application Ser. No. 63/170,512 filed Apr. 4, 2021, which is herein incorporated by reference in its entirety.

### FIELD

The present disclosure generally relates to electric field therapy, and in particular, to a system and associated method for a machine-learning guided planning environment for optimizing electric field therapy treatment parameters on a case-by-case basis.

### BACKGROUND

Cancer has risen to become the paramount medical dilemma for the aging population and the advancement of science and technology have provided novel methods for delivering therapy. Glioblastoma, the most common primary brain malignancy, is one such cancer that has been targeted by an innovative technology implementing alternating electric fields, referred to as Tumor Treating Field therapy.

Antimitotic effects of alternating electric field (AEF) are thought to originate through a multitude of disrupted physiologic processes: DNA repair, autophagy, cell migration, permeability, and immunological responses. These effects are likely related to the AEF impact on polarizable molecules through the accentuation of dipolar charges. Studies have demonstrated a direct correlation between low doubling time for cancerous cell lines (high division rate) and a high rate of AEF induced cell death. This effect is specific to cell type such that certain cancer cell types are shown to respond to certain ranges of AEF frequency and magnitude, with additive efficacy being contributed by greater coverage of AEF 3-dimensional orientations. Therefore, AEF magnitude and orientation are highly variable dependent on the target tissue of interest and geometry of the delivering device, requiring tailoring to optimize the delivery of this therapy. The dose of AEF therapy as estimated by finite element modeling has demonstrated that the dose of AEF (referring to magnitude of electric field, V/cm, time-lapsed orientations of the electric field, and the duration of treatment) is a critical component to the efficacy of the therapy. Measurement of AEF within body tissue is challenging to accomplish, given the requirement for multiple electrodes within the tissue and the gradient nature of an electric field distribution. The knowledge of the “dosage” of AEF that is feasible based on an electrode configuration, stimulating voltage, and body tissue type has immense value in treatment planning purposes, and for monitoring of the maintenance of therapeutic stimulation.

Predictive electrical property modeling (i.e. volume conductor modeling) can be performed for individual patients; however, this is time consuming and challenging to perform on individual patients, due to the need to use the patients individual imaging and complete mesh segmentation of the tissue subtypes.

It is with these observations in mind, among others, that various aspects of the present disclosure were conceived and developed.

### BRIEF DESCRIPTION OF THE DRAWINGS

FIGS. 1A and 1B are simplified diagrams showing an electric field therapy (EFT) planning system that accepts patient imaging as input and enables a practitioner to plan EFT treatment based on patient-specific parameters;

FIG. 2 is a simplified diagram showing a mesh modeling system of the EFT planning system of FIGS. 1A and 1B;

FIGS. 3A-3D are a series of images showing creation of a 3D virtual space mesh model using radiographic patient imaging slices;

FIG. 4 is a simplified diagram showing an EFT optimization system of the EFT planning system of FIGS. 1A and 1B;

FIG. 5 is a simplified illustration showing volumetric definition of a region of interest and modeled electrode objects within a 3D virtual space according to the EFT planning system of FIGS. 1A and 1B;

FIGS. 6A-6C are a series of simplified illustrations showing an example visualization environment according to the EFT planning system of FIGS. 1A and 1B;

FIG. 7 is a simplified diagram showing an EFT optimization reference library of the EFT optimization system of FIG. 4;

FIG. 8 is a simplified diagram showing an “optimize electrode configuration” module of the EFT optimization system of FIG. 4 with respect to a first grouping of variables of interest;

FIG. 9 is a simplified diagram showing an “optimize electrode configuration” module of the EFT optimization system of FIG. 4 with respect to a second grouping of variables of interest;

FIG. 10 is a simplified diagram showing a “resection region check” module of the EFT optimization system of FIG. 4;

FIG. 11 is a simplified diagram showing a “optimize stimulating parameters” module of the EFT optimization system of FIG. 4;

FIG. 12 is a simplified diagram showing incorporation of real-world measured data and resultant correction of EFT optimization parameters of the EFT optimization system of FIG. 4;

FIG. 13A is a simplified process flow showing an EFT planning process according to the EFT planning system of FIGS. 1A and 1B;

FIG. 13B is a simplified process flow showing various sub-steps of the EFT planning process of FIG. 13B;

FIG. 14 is a simplified diagram showing an exemplary computing system for implementation of the EFT planning system of FIGS. 1A and 1B; and

FIG. 15 is a simplified diagram showing an example neural network architecture model for implementation of aspects of the EFT planning system of FIGS. 1A and 1B.

Corresponding reference characters indicate corresponding elements among the view of the drawings. The headings used in the figures do not limit the scope of the claims.

### DETAILED DESCRIPTION

The present disclosure describes systems and methods for a computer-implemented electric field therapy (EFT) planning system that uses machine learning to guide and optimize a set of EFT treatment parameters based on patient imaging. In particular, the EFT planning system uses machine learning principles to educate a system that provides tissue segmentation and meshing based on radiographic imaging to permit generation of a virtual patient-specific electric field map of the tissue to aid surgical implantation or treatment planning of one or more implantable electrodes. Once implanted within tissue, the electrodes can also provide real-world electric field strength data which can provide feedback to the system. The system can also adopt machine learning principles for optimizing the set of EFT treatment parameters including stimulating parameters (e.g., waveform parameters) and implant type and positioning parameters, and can provide a pre-operative visual simulation of the effects of one or more selected EFT treatment parameters of the set of EFT treatment parameters. Overall, the knowledge of case-specific electric field distribution within tissue will permit advanced treatment planning to modulate and optimize the efficacy of EFT treatment.

### Overview

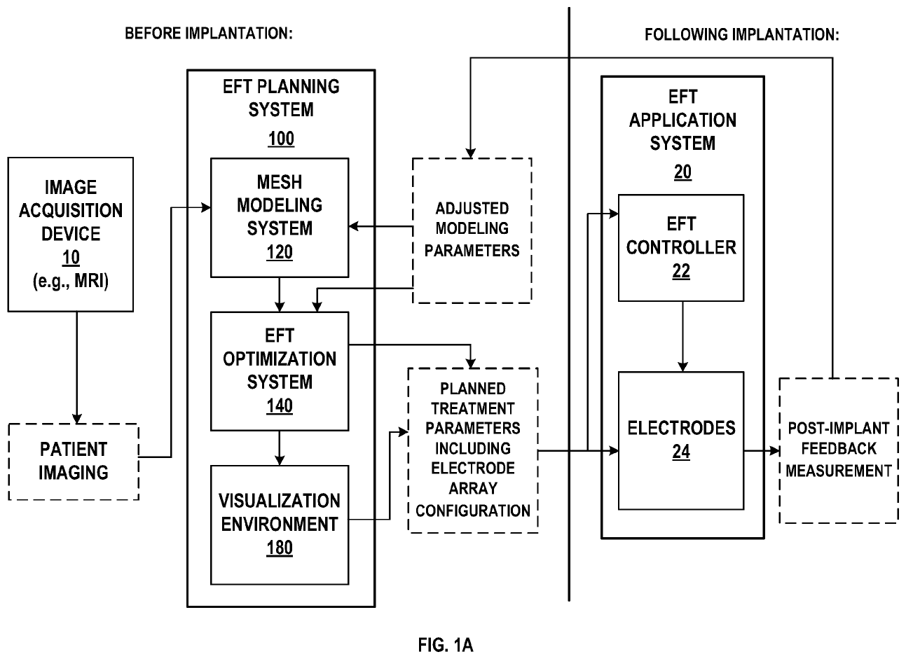

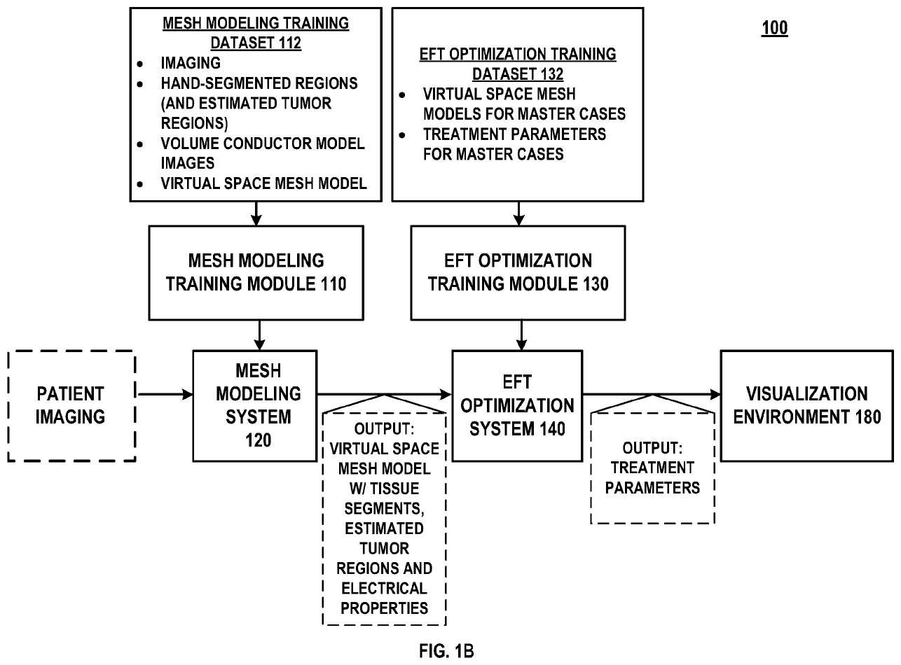

Referring to FIGS. 1A and 1B, an EFT planning system 100 (denoted herein as “system 100”) is provided for planning and optimizing a set of EFT treatment parameters of an implantable EFT application system 20 based on patient-specific imaging data, such as cross-sectional magnetic resonance imaging (MRI) data obtained from an image acquisition device 10. As illustrated, the EFT planning system 100 is operable to import patient imaging from the image acquisition device 10 into the EFT planning system 100 in the form of one or more cross-sectional image “slices” such as those obtained through an MRI imaging sequence. The EFT planning system 100 aids the practitioner in determining the set of EFT treatment parameters including an electrode array configuration for patient-specific application of EFT that result in a total area of effect of the implantable EFT application system 20 reaching a sufficient coverage threshold across the region of interest. Following implantation of the implantable EFT application system 20 including one or more electrodes 24 into tissue according to the set of EFT treatment parameters, the EFT planning system 100 can incorporate post-implant feedback measurements from one or more electrodes 24 to improve modeling.

The EFT planning system 100 includes a mesh modeling system 120 that uses a machine learning based model to generate a case-specific 3-D virtual mesh model of patient anatomy that includes segmented tissue areas and associated volumetric electrical properties for each segmented tissue area distinguishable within the patient imaging data. The EFT planning system 100 also includes an EFT optimization system 140 that optimizes the set of EFT treatment parameters based on the case-specific 3-D virtual mesh model including stimulating parameters (e.g., applied voltage or current amplitudes, waveform frequency, and waveform shape) and electrode configurations (e.g., electrode types, contact configurations for each electrode, electrode count, and electrode positions). Further, the EFT planning system 100 provides a visualization environment 180 in communication with the mesh modeling system 120 and the EFT optimization system 140 that enables a practitioner to simulate and view the effects of various EFT treatment parameters of the set of EFT treatment parameters with respect to the 3-D virtual mesh model of patient anatomy. In some embodiments, the visualization environment 180 can serve as a user interface that enables a practitioner to control various parameters and variables that are simulated to customize EFT treatment on a case-by-case basis.

The EFT planning system 100 uses imaging data that is commonly acquired during a patient's normal clinical course (MRI T1 and/or T2 weighted sequences) for the purposes of machine learning-guided anatomical segmentation of the 3-D virtual mesh model and subsequent assignment of electrical properties. These electrical properties could be assumed to be isotropic for the purposes of representing the anatomical structure or be considered anisotropic. This 3-D virtual mesh model with segmented tissue areas and associated volumetric electrical properties then permits finite element modeling analysis through the EFT optimization system 140 for subsequent evaluation of the set of EFT treatment parameters such as implant placement, extent of necessary tumor removal to achieve therapeutic minimums, electrical stimulatory parameter assignment, and modulatory activities. The optimal set of EFT treatment parameters result in an area of effect of the one or more electrodes reaching a sufficient coverage threshold across the region of interest.

Following determination of the set of EFT treatment parameters using the EFT planning system 100, the set of EFT treatment parameters including an electrode array configuration can be applied to the patient by the implantable EFT application system 20 including an EFT controller 22 in communication with the one or more electrodes 24, which are surgically implanted within the body to apply EFT treatment. In some embodiments, the one or more electrodes 24 can measure one or more post-implant feedback values from within the tissue and communicate the post-implant feedback to the EFT planning system 100 to adjust one or more modeling parameters of the EFT planning system 100 and improve the EFT planning system 100 overtime.

Referring to FIG. 1B, the mesh modeling system 120 can include one or more machine learning models trainable through a mesh modeling training module 110 that essentially “teaches” the mesh modeling system 120 to: (1) segment tissue types present within the patient imaging; and (2) correlate one or more volumetric electrical properties with each segmented tissue type present within the patient imaging. The mesh modeling training module 110 can use a set of mesh modeling training dataset 112 that includes data from a plurality of training cases, each training case including at least one of: (1) training case-specific imaging data (e.g. MRI image slices for a training case); (2) hand-segmented tissue regions defined with respect to the training case-specific imaging data; (3) volume conductor imaging data defined with respect to the training case-specific imaging data; and (4) a resultant virtual space mesh model for each training case. The hand-segmented tissue regions identify various tissue types present within the training case-specific imaging data, which are expected to have their own electrical properties based on tissue composition differences relative to the surrounding tissues. The volume conductor imaging data provides empirical data showing volumetric electrical properties for various segmented locations within tissue and can be correlated directly to the training case-specific imaging data.

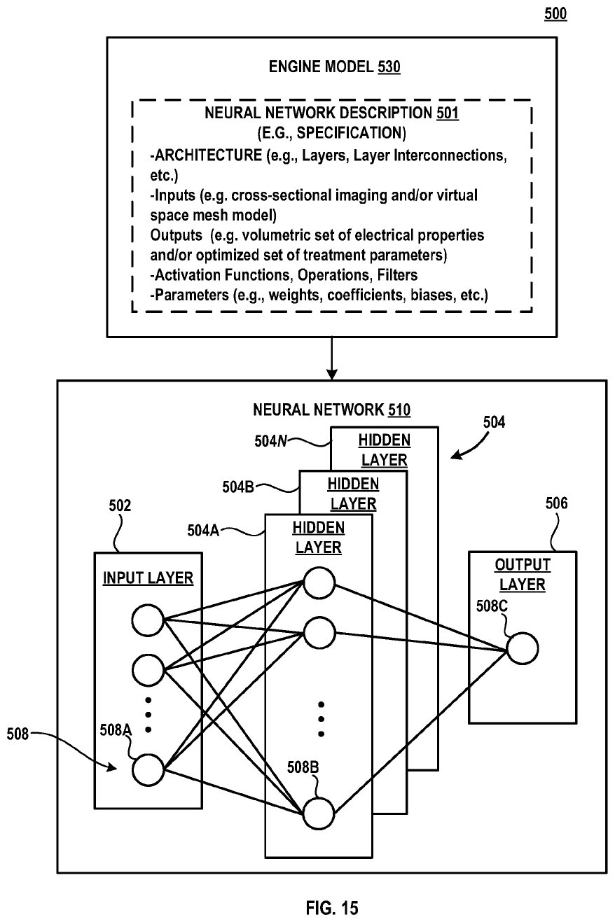

Based on correlations between the training case-specific imaging data, resultant hand-segmented tissue regions and corresponding volume conductor modeling imaging data, in some embodiments the mesh modeling training module 110 trains the mesh modeling system 120 to segment tissue types and anatomical structures present within patient imaging. Further, in some embodiments, the mesh modeling training module 110 also trains the mesh modeling system 120 to correlate one or more electrical properties with the segmented tissue types and structures identified within the patient imaging. As such, the mesh modeling system 120 can accept case-specific patient imaging and generate a resultant 3D virtual space mesh model that is case-specific, maintains knowledge of discrete anatomical structures and tissue segments within the 3D virtual space mesh model, and provides annotations including estimated electrical properties for various tissue types and structures identifiable within the patient imaging. The outputs of the mesh modeling system 120 can be facilitated by a plurality of possible machine learning methods including supervised, unsupervised, or reinforcement learning environments, and can be structured according to examples outlined in FIG. 15.

Alternatively, in some embodiments the mesh modeling system 120 can adopt an imaging modality which provides direct assessment of tissue/organ dielectric properties such as the use of magnetic resonance imaging impedance tomography. This pathway does not require the immediate adoption of a machine learning model for the purposes of generating a virtual model that represents the tissue/organ with assigned dielectric properties. However, the application of a machine learning model could be used to generate discrete virtual mesh volumes (tissue segments) described above and then assigning then anisotropic properties directly surmised from the magnetic resonance imaging impedance tomography (for example). Note that discrete anatomical structures are not required for the impendence tomography technique, in stark contrast to the anatomically based approach to dielectric property mapping based on segmented tissue types and anatomical structures as described above.

The EFT optimization system 140 can also include one or more machine learning models trainable through an EFT optimization training module 130 that essentially teaches the EFT optimization system 140 to determine the set of EFT treatment parameters based on the 3D virtual space mesh model for the specific case that result in sufficient coverage of the region-of-interest identifiable within the virtual space mesh model. The EFT optimization training module 130 can use a set of EFT optimization training data 132 that includes data from a plurality of training cases, each training case including at least one of: virtual space mesh models for a plurality of medically-reviewed “master” cases and sets of EFT treatment parameters for the plurality of medically-reviewed “master” cases. As mentioned above and as will be discussed in further detail herein, the EFT optimization training module 130 essentially “teaches” the EFT optimization system 140 to model the effects of various EFT treatment parameters of the set of EFT treatment parameters within tissue based on the tissue properties present within the virtual space mesh model, and to optimize the set of EFT treatment parameters based on the modeled effects. The set of EFT treatment parameters can include stimulating parameters to be applied to the tissue by the implantable EFT application system 20, in addition to electrode configuration parameters such as electrode type, electrode count, electrode position, and electrode design parameters. Additionally, the EFT optimization training module 130 can incorporate empirical feedback following implantation to compare expected results with actual measured results and can update various modeling and/or optimization parameters of the EFT optimization system 140 accordingly. The outputs of the EFT optimization system 140 can be facilitated by a plurality of possible machine learning methods including supervised, unsupervised, or reinforcement learning environments, and can be structured according to examples outlined in FIG. 15.

The visualization environment 180 can communicate directly with the EFT optimization system 140 to display a 3D virtual space including various virtual models including the virtual space mesh model, a virtual model of a region-of-interest (ROI), virtual models of the one or more electrodes 24 of the implantable EFT application system 20 and resultant modeled areas-of-effect (AOEs) throughout the virtual space mesh model. The visualization environment 180 can act as a user interface to enable the practitioner to control simulations and enter values or ranges for various EFT treatment parameters of the set of EFT treatment parameters for optimization by the EFT optimization system 140.

While several examples are provided herein with respect to a human brain as the structure-of-interest, it should be noted that aspects of this disclosure are also applicable to any parenchymal structure and associated extra-parenchymal or intra-parenchymal tissues for application of EFT within the body.

### Mesh Modeling System

Constructing a Virtual Space Model with Volume-Based Electrical Properties

Volume conductor modeling (VCM) permits the estimation of electric field distribution within finite-element modeled tissue to estimate local electric field strength and momentary or time-lapsed field orientation of an applied electric field dependent on the conductivity and permittivity values of the tissue of interest, following the stimulation of tissue by implanted electrodes. In one example with respect to a human brain, various components of brain tissue can be individually segmented within a VCM model of the brain (given that they each possess a unique conductivity and permittivity value). With a VCM model, one can more accurately predict the distribution of electric field within tissue and can estimate a dose of EFT that would be experienced by various regions within the brain dependent upon the VCM model alone. However, application of VCM simulation on a patient-by-patient basis would not be feasible given the time-consuming nature of manually segmenting a model for VCM simulation. To overcome this hurdle in electric field estimation, the EFT planning system 100 uses imaging technology such as MRI images to serve as a reference for the tissue structure of interest to estimate segmented tissue areas including anatomical structures and estimate associated volumetric electrical properties accordingly. Patient-specific radiographic imaging data provides critical anatomical novelties that inevitably will impact the resulting AEF distribution.

To convert the imaging data to a clinically relevant estimation of electric field, mesh modeling system 120 includes one or more machine learning models (following iterative exposures to the set of mesh modeling training dataset 112 segmented by human oversight) to enable the mesh modeling system 120 to predictively segment subsequent imaging of similar anatomical nature. Following the generation of a segmented volumetric model of the tissue of interest, pre-defined values for conductivity and permittivity within the tissue subtypes can be applied. Alternatively, a virtual mesh model with volumetric electrical properties represented in voxel dimensions can be acquired through magnetic resonance imaging impedance tomography.

### Generating the Mesh Modeling Training Dataset

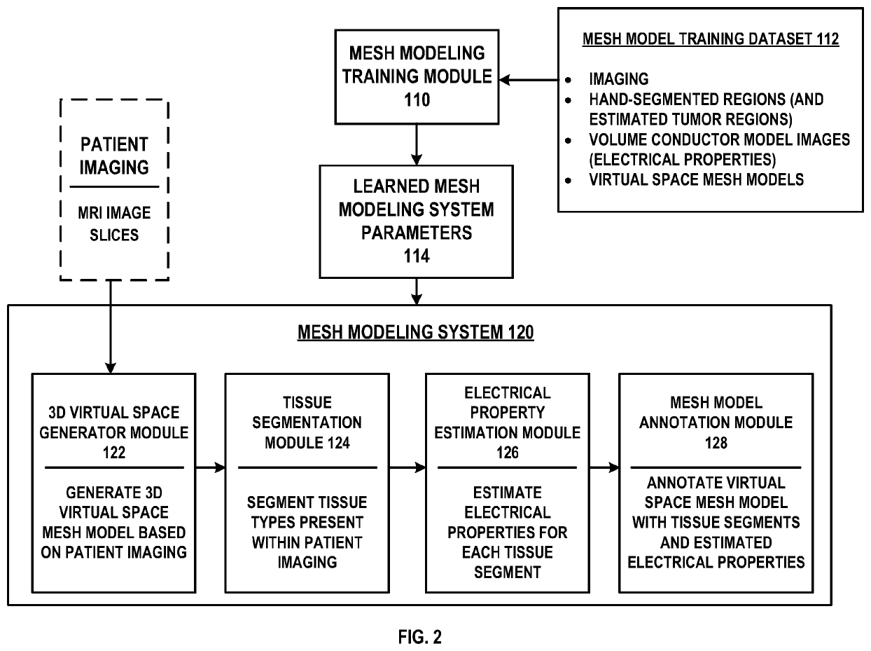

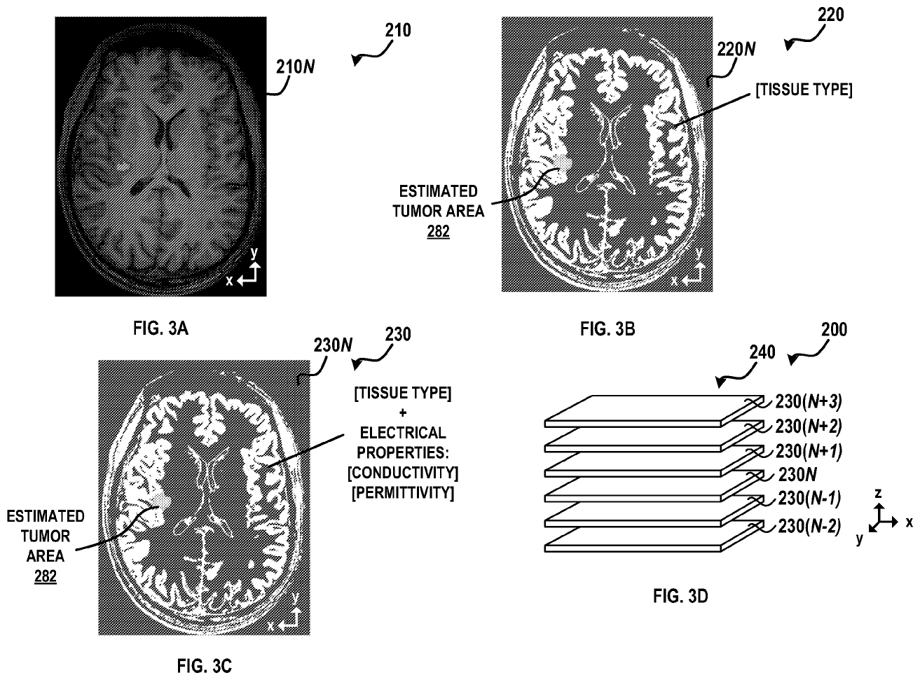

Referring to FIGS. 2 and 3, a process of generating the mesh modeling training dataset 112 begins with initial image processing of training case cross-sectional imaging data that facilitates subsequent analysis. These steps include initial acquisition of some form of cross-sectional imaging of a plurality of test cases, for example, of an MRI T1 or T2 sequence to permit anatomical resolution of the tissue or organ of interest. Next, the cross-sectional imaging data must be made available within a virtual software environment that permits division of objects into manageable pieces (i.e. voxels) that are represented within a cartesian coordinate system (either in a virtual space or software representation of physical space). This can be achieved through a 3D Virtual Space Generator module 122 that generates a 3D virtual space mesh model based on the provided cross-sectional imaging data, which can be applied to training cases or to a specific case to be analyzed. Each voxel must then be assigned a correlative physical size which is variable based on parameters of the image acquisition device 10 (FIG. 1A). Voxels are represented in virtual space as an associated grayscale image value for pixel brightness as quantified by an 8-bit integer ranging from 0 to 255. Windowing of voxel intensities can be used to isolate anatomically contiguous regions of interest within the tissue/organ within the lower and upper cutoffs (0-255). Within the brain, for example, this contiguity analysis can be further complicated through the incorporation of structural detail acquired through diffusion tensor imaging (DTI). This additional modality of magnetic resonance imaging provides a means for radiographic assessment of structural continuity within the more encompassing tissue subtypes, such as “white matter”, that allows anatomical sub-structures such as individual fiber bundles or deep brain nuclei to be segregated from an otherwise homogenous voxel intensity object. While the above discussion is in terms of the brain as an organ of interest, this process can be applied to other structures within the body as well. The 3D Virtual Space Generator module 122 can be used for both generating the 3D virtual space mesh models for the training cases or generating a 3D virtual space mesh model for a patient to be analyzed as will be discussed in greater detail below.

Following the completion of these imaging processing tasks, to generate the mesh modeling training dataset 112, human-mediated virtual mesh segmentation is undertaken for the establishment of learning cases to be assessed by the machine learning algorithm. This human-mediated virtual mesh segmentation is used to define a library of anatomically isolated structures within tissues/organs of interest across a multitude of training case examples which demonstrate subtle or significant anatomical variability, which can be stored as part of the mesh modeling training dataset 112 and optionally within a set of learned mesh modeling system parameters 114 resultant of the training process. The result of the training process for tissue segmentation enables a tissue segmentation module 124 of the mesh modeling system 120 to segment tissue types including anatomical structures present within patient imaging and apply the segmented tissue types to each respective 3D virtual space mesh model. In some embodiments, the mesh modeling dataset can include images that include tumors or other cancerous tissue with associated identifiers to enable the mesh modeling system 120 to provide a volumetric estimated tumor region.

To further add to the mesh modeling training dataset 112, VCM is then applied for each training case to estimate electric field distributions within finite element modeled tissue to estimate the local field strength and field orientation dependent on the conductivity and permittivity values of the tissue of interest. This provides measurements of various electrical properties for each segmented tissue type present within the training cases, which can be stored as part of the mesh modeling training dataset 112 and optionally within a set of learned mesh modeling system parameters 114 resultant of the training process. The result of the training process for electrical property estimation enables an electrical property estimation module 126 of the mesh modeling system 120 to estimate a set of volumetric electrical properties for various segmented tissue types present within patient imaging, including tumors or other cancerous tissue.

### Training the Mesh Modeling System

Following human-derived population of the mesh modeling training dataset 112 for the machine learning application, test cases can be input via the exposure of unsegmented radiographic imaging of a tissue/organ of interest to the mesh modeling system 120 through the mesh modeling training module 110 to provide the set of learned mesh modeling system parameters 114 to the mesh modeling system 120. A similar approach to that described within the manual human mediated segmentation step is employed by the tissue segmentation module 124 to estimate anatomical mesh segmentation borders present within the test case imaging. This would include utilization of voxel intensity ratios across a single imaging modality (i.e., MRI T1 without contrast sequence) or multiple imaging modalities (i.e., MRI T1 without contrast+T1 with contrast+T2 without contrast+computed tomography). Additionally, voxel intensity-independent strategies such as those involving DTI can be considered to permit isolation of component structures within larger parent structures (for example, superior longitudinal fasciculus within the larger structure of cerebral white matter). This level of granular detail can permit isolation of otherwise homogenously appearing structures within simple non-tensor voxel intensity comparisons. The output of the tissue segmentation module 124 can be facilitated by a plurality of possible machine learning methods including supervised, unsupervised, or reinforcement learning environments, and can be structured according to examples outlined in FIG. 15. As such, the tissue segmentation module 124 can be trained to segment tissue types present within patient imaging, including identification of an estimated tumor area 282 within each cross-sectional image as part of its tissue segmentation task.

Further, the electrical property estimation module 126 can be similarly trained and employed to estimate the set of volumetric electrical properties across the plurality of tissue segments. For training, test cases can be input via the exposure of unannotated radiographic imaging of a tissue/organ of interest to the mesh modeling system 120 through the mesh modeling training module 110 to provide the set of learned mesh modeling system parameters 114 to the mesh modeling system 120. Based on the mesh modeling training dataset 112 and the set of learned mesh modeling system parameters 114, the electrical property estimation module 126 of the mesh modeling system 120 is trained to associate various electrical properties with tissue sub-types identified within the test case imaging through iterative exposure. The output of the electrical property estimation module 126 can be facilitated by a plurality of possible machine learning methods including supervised, unsupervised, or reinforcement learning environments. As such, the electrical property estimation module 126 can be trained to estimate the set of volumetric electrical properties using segmented tissue types present within patient imaging.

### Mesh Modeling System Outputs

Collectively, when applied to a patient case, the outputs of the tissue segmentation module 124 and the electrical property estimation module 126 of the mesh modeling system 120 represent a volumetric patient-specific virtual representation of tissue/organ anatomy that is either isotropically or anisotropically segmented with electrical property assignments. Used in combination with the 3D Virtual Space Generator Module 122 that forms a 3D virtual space mesh model object based on imported patient imaging slices, and a mesh model annotation module 128 that adds tissue segmentation data and a volumetric set of electrical properties from the tissue segmentation module 124 and the electrical property estimation module 126 to the 3D virtual space mesh model object, the mesh modeling system 120 generates a fully annotated 3D virtual space mesh model representative of case-specific anatomy.

This process is illustrated in FIGS. 3A-3D with respect to a cross-sectional imaging “slice” 210N of FIG. 3A, where cross-sectional imaging slice 210N is an MRI image slice, particularly an N^{th} slice of a plurality of slices of an MRI sequence taken by image acquisition device 10 (FIG. 1A) for a particular case. As illustrated, the cross-sectional imaging slice 210N defines a 2-D plane. FIG. 3B shows a simplified example of a segmented imaging “slice” 220N, which is a 2-D virtual space object based on the associated imaging “slice” 210N of FIG. 3A having been subjected to tissue segmentation obtained through voxel intensity thresholding from the imaging “slice” 210N as performed by the tissue segmentation module 124 of the mesh modeling system 120. As discussed above, the tissue segmentation module 124 can be machine-learning guided (i.e., the tissue segmentation module 124 learns to segment tissue types based on hand-segmented tissue data). Further, the tissue segmentation module 124 can identify an estimated tumor area 282 within one or more segmented imaging “slices” 220.

FIG. 3C shows a simplified example of an electrical property-annotated imaging “slice” 230N, which is a 2-D virtual space object based on the associated imaging “slice” 220N of FIG. 3B having been subjected to electrical property estimation obtained through electrical property estimation module 126 of the mesh modeling system 120. As discussed above, the electrical property estimation module 126 can be machine-learning guided (i.e., the electrical property estimation module 126 learns to correlate electrical properties with segmented tissue based on volume conductor modeling data and corresponding hand-segmented tissue data).

FIG. 3D shows an example combination of a plurality of electrical property-annotated imaging slices 230 (denoted in the example as “230 (N−2)” through “230 (N+3)”, although hundreds or thousands of slices can be included, depending on the settings of the image acquisition device 10 (FIG. 1A)) organized according to their respective locations in a 3D space to form a virtual space mesh model 240, which is a 3D virtual object in a 3D virtual space 200 representative of the cross-sectional patient imaging obtained through T1, T2, or DTI magnetic resonance imaging methods or another cross-sectional imaging method. The virtual space mesh model 240 can include various volumetric tissue segments and associated annotations for the volumetric set of electrical properties and tissue segments identified through the tissue segmentation module 124 and the electrical property estimation module 126. As will be discussed in greater detail below, the virtual space mesh model 240 can be incorporated within the 3D virtual space 200 for simulation and viewing by the visualization environment 180 and will further be used by the EFT optimization system 140 for optimizing the set of EFT treatment parameters.

### EFT Optimization System

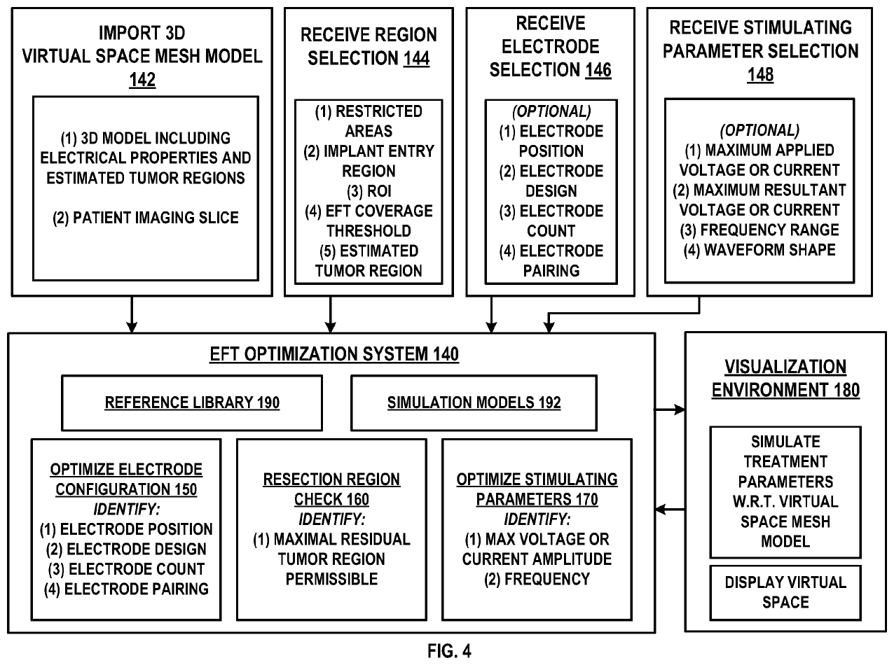

Referring to FIG. 4, the EFT optimization system 140 enables optimization of the set of EFT treatment parameters for case-specific application of EFT treatment to patient anatomy, the set of EFT treatment parameters including electrode configuration, a required resection region, and one or more stimulating parameters that dictate waveforms for application of EFT treatment. As shown, the EFT optimization system 140 requires several inputs including: (1) the virtual space mesh model 240; and (2) one or more region selections with respect to the virtual space mesh model 240. The EFT optimization system 140 communicates with an “import virtual space mesh model” block 142 that imports the virtual space mesh model 240 descriptive of anatomy of a particular patient, including a plurality of cross-sectional imaging slices 210 and volumetric set of electrical properties in addition to an estimated tumor region 280 defined within the virtual space mesh model 240. The estimated tumor region 280 can be a volumetric region constructed in the 3D virtual space 200 by summation of each respective estimated tumor area 282 identifiable within patient imaging (FIGS. 3B and 3C). It should be noted that while the virtual space mesh model 240 can be generated through the mesh modeling system 120, the virtual space mesh model 240 imported into the EFT optimization system 140 can be hand-modeled by the practitioner or acquired at least in part through impedance tomography. Further, the EFT optimization system 140 communicates with a “receive region selection” block 144 that enables receipt of one or more region parameters specified by the practitioner. In particular, the practitioner needs to select at least one volumetric region of interest (ROI) to be targeted by EFT treatment within the context of the virtual space mesh model 240, which can be modeled as an ROI object 250 defining a 3-dimensional volumetric region within the virtual space mesh model 240 that is targeted for EFT treatment. The practitioner can also approve or modify the estimated tumor region 280. The estimated tumor region 280 would ideally be within a region-of-interest (ROI) object 250, although it should be noted that some areas of the ROI object 250 can also extend to surrounding tissue beyond the estimated tumor region 280 (i.e. a peri-tumoral region or tumor bed at least 3 mm surrounding the estimated tumor region and other nearby anatomical structures). This may be achieved within the visualization environment 180, which can act as a user interface for the practitioner to enter/specify various simulation parameters or the set of EFT treatment parameters, import relevant data, and display results. Alternatively, the practitioner can select region parameters including the ROI, an implant entry region, one or more restricted areas, etc., from a listing of one or more structures or segmented tissue areas identified within the virtual space mesh model 240. Further, the practitioner can also select one or more “optimal” threshold parameters for EFT coverage across the ROI, such as target electric field intensity values and time-lapsed orientation coverage (such as full spherical field orientations coverage with a 20 degree binning threshold such that no orientation gaps are present within a multitude of 20 degree zones along a spherical virtual space) within the ROL.

Optionally, the practitioner can enter one or more electrode configuration selections through a “receive electrode selection” block 146. These can include electrode configuration parameters such as electrode position, electrode design, and electrode count. While the EFT optimization system 140 enables optimization of the electrode configuration as will be described in greater detail below, the practitioner can input initial values or ranges that can be fixed constants during the optimization process or can serve as starting points for the optimization process. Similarly, the practitioner can enter one or more stimulating parameter selections through a “receive stimulating parameter selection” block 148. These can include stimulating parameters including maximum applied voltage or current (i.e., amplitude of an applied waveform), maximum resultant voltage or current (i.e., intended effect within the tissue), applied waveform frequency, and a waveform shape (e.g., square, sine, sawtooth, ramp, etc.). While the EFT optimization system 140 enables optimization of the stimulating parameters as will be described in greater detail below, the practitioner can input initial stimulating parameter values that can be fixed constants during the optimization process or can serve as starting points for the optimization process. The EFT optimization system 140 can hold one or more selected values constant while optimizing other values or while simulating the effect on tissue.

If no electrode configuration selections or stimulating parameter selections are provided, then the EFT optimization system 140 can perform one or both of the following: (1) provide a machine-learning guided estimation of parameters based on similarity to one or more “master cases” located within the EFT optimization training data 132 (FIG. 1) and/or a reference library 190; and (2) iteratively sweep parameters until optimal solution is found. In one example combining both options, the EFT optimization system 140 can use one or more machine learning models to select one or more starting electrode configuration parameters or stimulating parameters based on a similar master case, and then sweep the electrode configuration parameters or stimulating parameters across a range to “tweak” the values until they are optimized.

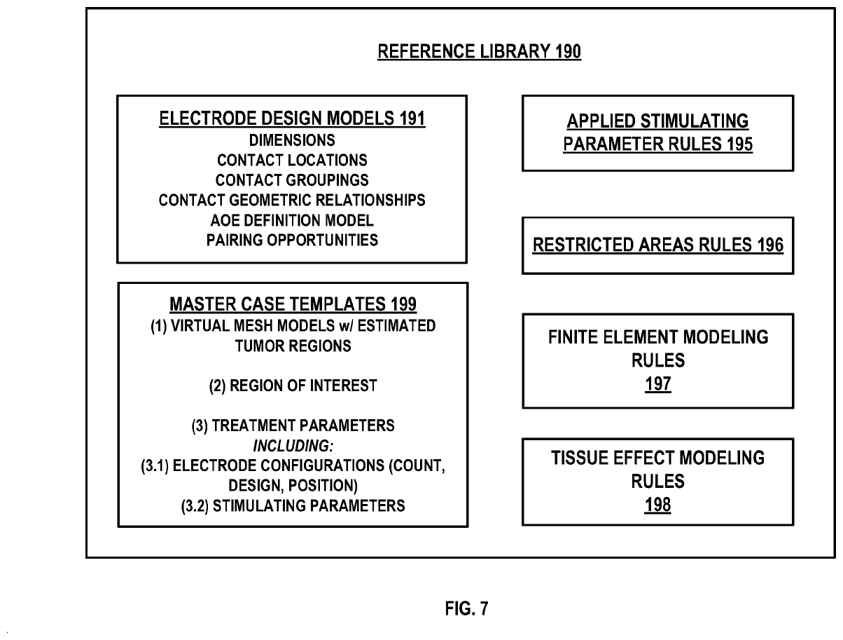

As further shown, the EFT optimization system 140 includes one or more simulation modules 192 including a finite element modeling module 193 (FIGS. 7-11) that models virtual hardware components corresponding to the one or more electrodes 24 (FIG. 1A) to be implanted based on the set of EFT treatment parameters including selected stimulating parameters. The one or more simulation modules 192 also include a tissue effect modeling module 194 (FIGS. 7-11) that models the effect of the virtual hardware components on tissue based on the virtual mesh model 240, which includes the volumetric set of electrical properties across tissue segments identified within imaging and further based on the virtual hardware components and the set of EFT treatment parameters. In some embodiments, the finite element modeling module 193 and the tissue effect modeling module 194 can communicate with one or more machine-learning models that learn model parameters for how a stimulating waveform applied through the one or more electrodes 24 represented by the virtual hardware components propagates through tissue; the learning process can be guided by EFT optimization training data 132 discussed above and/or can be feedback-guided by incorporating operating data obtained post-implantation. Resources for the finite element modeling module 193 and the tissue effect modeling module 194 can be located within the reference library 190 (FIG. 7) maintained by the EFT optimization system 140.

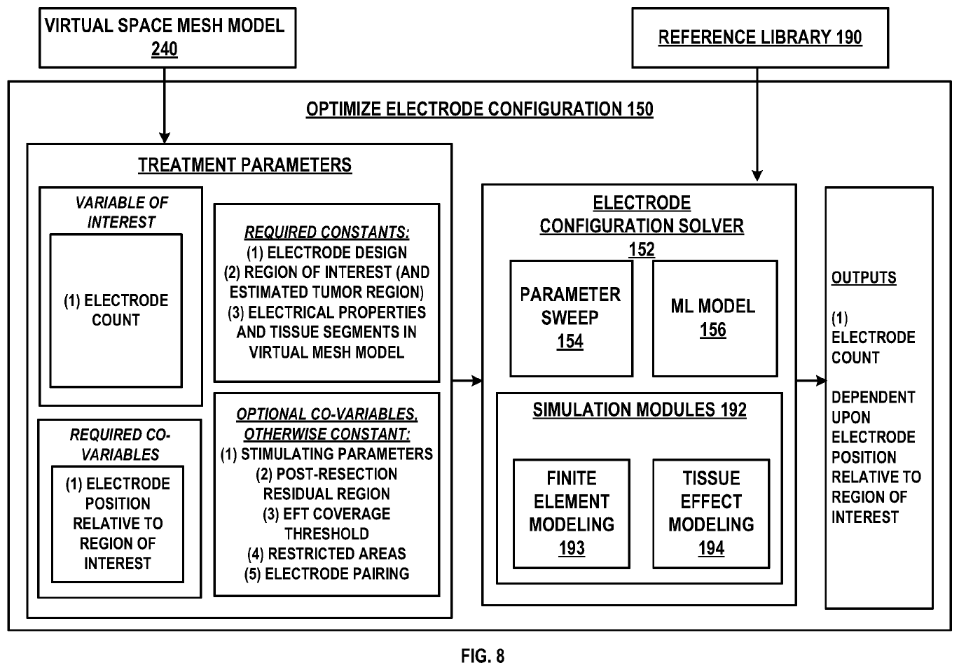

The EFT optimization system 140 can include a plurality of sub-modules that invoke the one or more simulation modules 192 to optimize the set of EFT treatment parameters based on the virtual space mesh model 240, including an “optimize electrode configuration” module 150 that optimizes the configuration of electrodes, a “resection region check” module 160 that identifies a maximal residual tumor region permissible based on the remaining EFT treatment parameters of the set of EFT treatment parameters, and an “optimize stimulating parameters” module 170 that optimizes various waveform parameters for application of EFT treatment through the plurality of electrodes. “Optimize electrode configuration” module 150, “resection region check” module 160 and “optimize stimulating parameters” module 170 will each be described in further detail below with reference to FIGS. 8-11.

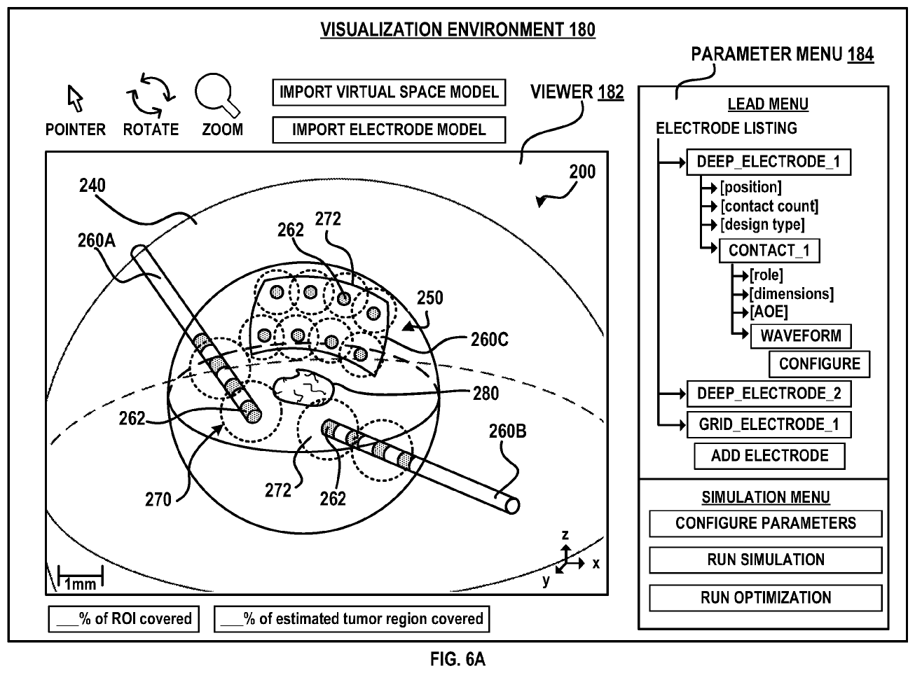

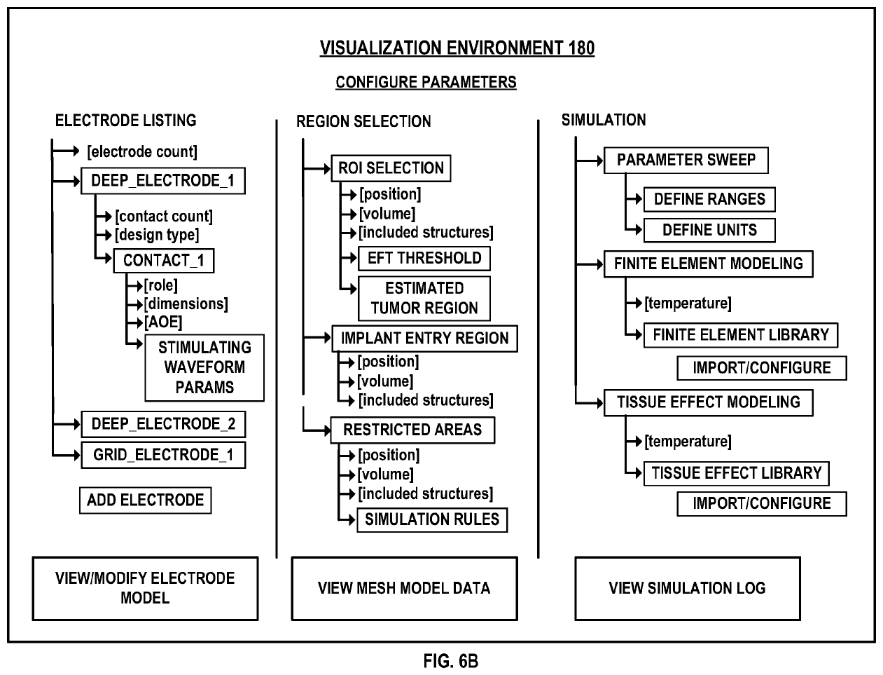

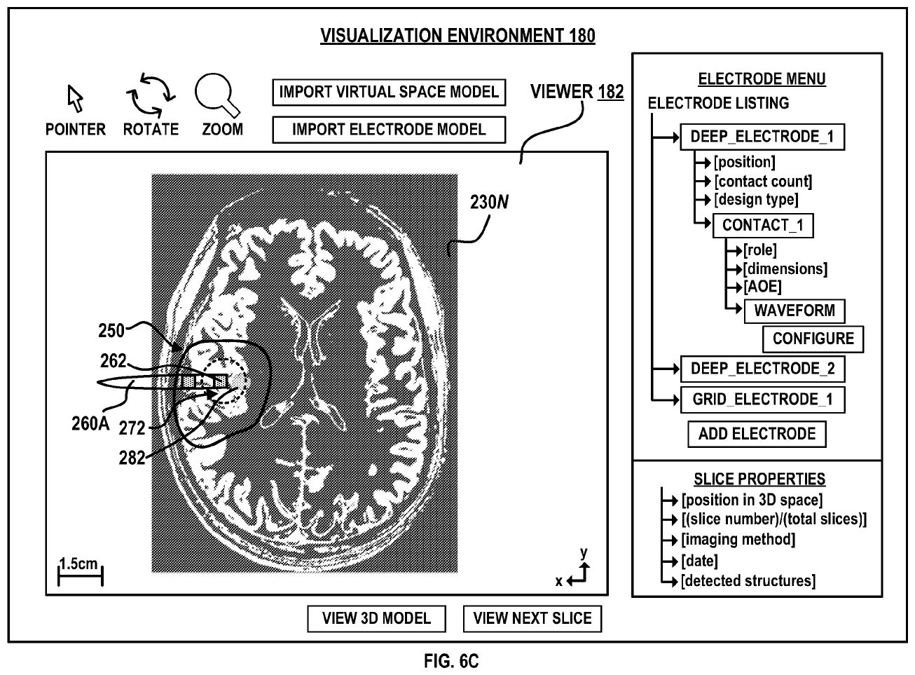

The EFT optimization system 140 also communicates with the visualization environment 180, shown in FIG. 6A and discussed in further detail below, that provides a virtual workspace to view and control the simulated application of EFT in the context of the virtual space mesh model 240, and can enable a practitioner to toggle values and view their effects. In some embodiments, inputs to the EFT optimization system 140 including the “receive region selection” block 144, the “receive electrode selection” block 146, and the “receive stimulating parameter selection” block 148 can be provided to the EFT optimization system 140 through the visualization environment 180.

### Volumetric Definitions and Virtual Space Effect Modeling

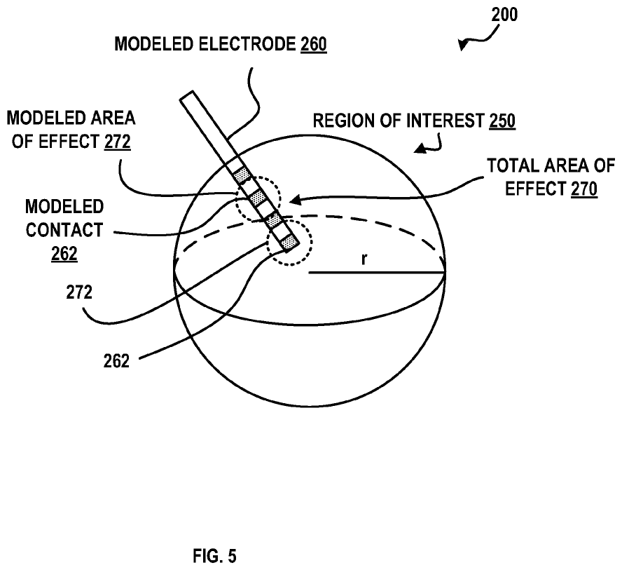

Referring to FIG. 5, the 3D virtual space 200 is illustrated that provides an example rendering of the ROI object 250, which would be located somewhere within the virtual space mesh model 240 (as shown in FIG. 6A). Further, the 3D virtual space 200 can also define the estimated tumor region 280 as identified through the cross-sectional imaging, which can be considered a volume summation of one or more estimated tumor areas 282 identified within the cross-sectional imaging by the tissue segmentation module 124 or by another means such as through direct input from the practitioner. The estimated tumor region 280 defines a 3-dimensional shape and total volume (with units such as cm³ or mm³) within the 3D virtual space 200, and a position relative to the ROI object 250. To reiterate, the estimated tumor region 280 would ideally exist within ROI object 250, although it should be noted that some areas of the ROI object 250 can also extend to surrounding (non-cancerous) tissue. The 3D virtual space 200 can also include one or more modeled electrode objects 260, each modeled electrode object 260 being a virtual model of an electrode of the one or more electrodes 24 of the EFT application system 20 stored in the reference library 190 (FIG. 7) that can be superimposed over the ROI object 250, inherently having “electrode design” parameters which can include number of contacts, dimensions, and resultant area of effect (given stimulating parameters). These electrode design parameters can be hand-selected or modified and/or optimized with the EFT optimization system 140 based on: (1) the position, tissue properties and volume of the ROI as provided through the “receive region selection” block 144 (FIG. 4) and as provided within the virtual space mesh model 240; and (2) EFT coverage threshold (i.e., a desired electric field magnitude and orientation to be applied within the region of interest) as provided through the “receive region selection” block 144. Additional electrode configuration parameters that can be provided by the practitioner and that are reflected within the 3D virtual space 200 include: (3) the position of each electrode relative to the ROI; and (4) electrode count (how many total electrodes to be applied).

Each modeled electrode object 260 can include one or more contact objects 262 positioned at different locations along the modeled electrode object 260. The contact objects 262 can vary in quantity, size, role, and location along the modeled electrode object 260 and these properties can be optimized by the EFT optimization system 140. modeled electrode Each contact objects 262 results in one or more resultant virtual area of effect (AOE) objects 272 modeled in the 3D virtual space 200 for each contact object 262 of the modeled electrode object 260. The AOE object 272 associated with each respective contact object 262 can be dependent on an AOE definition model stored in the reference library 190 that dictates how each respective AOE object 272 should be modeled during simulation according to the current set of treatment parameters. Each virtual area of effect (AOE) object 272 can be a volume of EFT coverage within tissue that result from the simulation depending upon the electrical properties of the associated contact object 262, applied stimulating parameters and the volumetric set of electrical properties of the tissue as dictated within the virtual mesh model 240. A total AOE region 270 is descriptive of a total “coverage zone” of sufficient EFT treatment relative to the region of interest and includes a summation of each respective AOE object 272 that meets the EFT coverage threshold of sufficient treatment as provided through the “receive region selection” block 144.

Referring briefly to FIG. 6A, the visualization environment 180 provides a visual user interface in communication with the EFT optimization system 140. The visualization environment 180 provides a viewer 182 that shows the 3D virtual space 200 including the virtual space mesh model 240, the estimated tumor region 280 and the virtual ROI object 250, as well as the one or more modeled electrode objects 260 and resultant virtual AOE objects 272.

The one or more simulation modules 192 use the one or more modeled electrode objects 260 as the virtual hardware models to model the effects of the one or more electrodes 24 throughout tissue according to one or more sets of EFT treatment parameters. In some embodiments, the one or more simulation modules 192 require at least an initialization of the set of EFT treatment parameters of the modeled electrode objects 260 including electrode configuration parameters (electrode design, electrode count and position of each modeled electrode object 260 relative to the virtual ROI object 250), and the stimulating parameters to be applied to the virtual space mesh model 240 by modeled electrode object 260 in order to identify the resultant total AOE region 270 for the set of EFT treatment parameters. Since the set of EFT treatment parameters are to be optimized by the EFT optimization system 140, the one or more simulation modules 192 can model the effects of the one or more electrodes 24 throughout tissue as the EFT optimization system 140 varies the set of EFT treatment parameters applied with the modeled electrode object 260 to identify the optimal set of EFT treatment parameters that result in the best modeled effects through the virtual space mesh model 240. In some embodiments, the optimal set of EFT treatment parameters result in a maximal resultant total AOE region 270 with respect to the ROI object 250 that meets the EFT coverage threshold of sufficient treatment while maintaining practitioner preferences (if one or more parameters are specified as “fixed constants” by the practitioner) and while not violating modeling rules (i.e., so as not to generate impossible configurations) or safety guidelines (i.e., applied stimulating parameter rules 195 or restricted areas rules 196).

### Example User Interface

Referring to FIGS. 6A-6C, an example user interface is provided within the visualization environment 180. As discussed above, the visualization environment 180 provides a viewer 182 that displays the 3D virtual space 200, including the imported virtual space mesh model 240 descriptive of patient anatomy and the ROI object 250 and estimated tumor region 280 that are subsets of the virtual space mesh model 240 and denote the target area for EFT application within patient anatomy. The viewer 182 also displays the one or more modeled electrode objects 260 including associated contact objects 262 relative to the ROI object 250. In the example shown, three modeled electrode objects 260A-C are illustrated within an example ROI object 250, including a first penetrating modeled electrode object 260A, a second modeled penetrating electrode object 260B and a third modeled surface electrode object 260C, each defining respective positions, orientations, and sets of design parameters, along with associated contact objects 262 and resultant AOE objects 272. The practitioner can view the configurations and simulation results of each respective modeled electrode object 260 relative to the ROI object 250 within the 3D virtual space 200.

The visualization environment 180 communicates with the simulation modules 192 to simulate the set of EFT treatment parameters with respect to the 3D virtual space 200, the results of which are visually displayed through the viewer 182. For optimization, the values of the set of EFT treatment parameters are varied through the EFT optimization system 140 as the simulation modules 192 are run to identify the optimal set of EFT treatment parameters that result in the best EFT coverage of the ROI, as simulated by the simulation modules 192.

The visualization environment 180 can additionally provide a parameter menu 184 that enables viewing and/or altering of various parameters, including the set of EFT treatment parameters to be optimized by the EFT optimization system 140. As shown, the parameter menu 184 can include a section to select a pre-defined geometry arrangement of the one or more modeled electrode objects 260 from a menu or to configure their own custom geometric arrangement, and a “electrode listing” section that enables a practitioner to view, add and change properties for each respective modeled electrode object 260 including contact assignments, electrode design parameters, and can also optionally configure one or more stimulation waveform parameters to be applied by each respective modeled electrode object 260. Electrodes can also be added to the 3D virtual space 200 through the parameter menu 184, with an option to import one or more additional modeled electrode objects 260 defining their own properties.

The parameter menu 184 can provide a simulation menu section that enables the practitioner to initiate a simulation based on present parameters, which may already be optimized by the EFT optimization system 140 or which may be entered by the user. The simulation menu section can also provide a “run optimization” option which would initiate an optimization sequence applied by the EFT optimization system 140 based on one or more initial EFT treatment parameters. This may include an additional “configure parameters” view that enables a practitioner to manage and select variables, including co-variables, fixing constants and optionally enables a practitioner to view the set of volumetric electrical properties and other mesh model data. An example is shown in FIG. 6B.

In addition, as shown in FIG. 6C, the visualization environment 180 can provide an option to view a 2D “slice” of an arrangement with a position of modeled electrode objects 260 and/or resultant AOE objects 272 superimposed over an MRI image slice of patient anatomy, translated back from the 3D virtual space to the 2D image slice space.

### Parameter Optimization Overview

Referring to FIGS. 4-11, the EFT optimization system 140 optimizes the set of EFT treatment parameters with respect to the virtual space mesh model 240 descriptive of anatomy of the patient and can incorporate various machine learning models and parameter sweep models to optimize the set of EFT treatment parameters through simulation such that a total expected area-of-effect of the applied EFT reaches a sufficient coverage threshold across the ROI object 250. In one aspect, inputs to the EFT optimization system 140 can include:
Modeled electrode objects 260 (including contact locations and wiring associations)
Patient-specific electrical property virtual objects (such as the annotated virtual space mesh model 240 acquired through mesh modeling system 120 or through hand-modeling)
Electrical impendence tomography generated virtual objects (ex. virtual space mesh model 240 at least in part acquired using impedance tomography)
A virtual map of the virtual ROI object 250 defined within the virtual space mesh model 240 (e.g., a tumor or a tumor bed)
Applied stimulating parameter rules 195 within a defined electric field therapy treatment system
Mesh volumes representing un-implantable or regions of restriction for implantation within the above mentioned virtual object(s) including restricted areas rules 196
Implant entry zone (for example, craniotomy for window of brain exposure)
The intended EFT coverage threshold (expected coverage zone region and electric field parameters such as intensity, direction, etc. across the virtual ROI object)

These inputs can be imported with the virtual space mesh model 240, stored within the reference library 190, and/or can be otherwise entered by the practitioner through a user interface which can include the visualization environment 180.

The desired outputs from the EFT optimization system 140 are the optimized set of EFT treatment parameters, including electrode configuration parameters which include:
Number of necessary electrodes of a single design type or a combination of different design types for a specific treatment environment (tissue/organ based on the ROI object 250)
Optimal electrode designs or a combination of different electrode designs to maximize therapeutic efficacy within the ROI object 250
Ideal electrode position within the virtual space to maximize either electric field therapy magnitude, electric field orientation, or to otherwise reach an expected coverage of electric field magnitude and electric field orientation within the ROI object 250
Optimized electrode pairing strategy within available contacts based on geometric position relative to the ROI object 250

Other optimized EFT treatment parameters of the set of EFT treatment parameters from the EFT optimization system 140 can include:
Defining the region within the ROI object 250 (ex. tumor coverage region) that is permissibly treated with a minimum threshold of EFT (field strength and field orientation) and displaying the region that is inadequately treated thereby defining the maximal residual region of the object of interest (ex. tumor) that can receive therapeutic treatment
Stimulating parameters including waveform parameters either through a voltage-controlled or current-controlled circuit across a complex system of electrode contacts, including cycling pairing combinations to enable optimized electric field therapy treatment within a region of interest in the virtual object volume

To optimize these outputs, the EFT optimization system 140 can use the simulation modules 192 including the finite element modeling module 193 and the tissue effect modeling module 194 to simulate the effects of the set of EFT treatment parameters with respect to the modeled electrode objects 260 and the virtual space mesh model 240. An optimal set of EFT treatment parameters results in sufficient coverage of electric field magnitude and electric field orientation across the ROI object 250. The set of EFT treatment parameters can be optimized with respect to the inputs through various machine learning models that provide estimations based on master cases and known effects of the set of EFT treatment parameters through tissue. Further, the outputs can optionally be optimized at least in part using one or more parameter sweep solver models that “sweep” treatment parameter values across various ranges during simulation by the simulation modules 192 to find the optimal set of EFT treatment parameters.

The EFT optimization system 140 can reference the reference library 190 of FIG. 7 when optimizing the set of EFT treatment parameters. For instance, the reference library 190 can include information for various electrode design models 191 that correspond with their respective modeled electrode objects 260, including dimensions, contact objects 262 and associated properties, locations, groupings, geometric relationships, AOE definition models and pairing opportunities for each modeled electrode object 260 (i.e., paired contacts that apply a waveform at complimentary phases or paired contacts configured in respective stimulating or measuring roles). Any of the above information for the electrode design models 191 can optionally be customized or modified by the practitioner. As mentioned above, the reference library 190 can include various circuit simulator resources including finite element modeling rules 197 and tissue effect modeling rules 198 respectively for the finite element modeling module 193 and the tissue effect modeling module 194. Further, the reference library 190 can include applied stimulating parameter rules 195 and restricted areas rules 196 that can provide medically reviewed safety guidelines for EFT treatment that are checked during simulation and optimization to make sure none are violated. Finally, the reference library 190 can include the one or more master case templates 199 that can be used to train one or more machine learning models of the EFT optimization system 140 and/or provide a similarity reference to the EFT optimization system 140 to select one or more starting EFT treatment parameters of the set of EFT treatment parameters to be swept across a range during optimization. The master case templates 199 can include medically reviewed virtual mesh models, associated ROI objects, and post-implantation sets of EFT treatment parameters including electrode configurations and stimulating parameters that can guide the EFT optimization system 140.

Optimization of each of the outputs will be individually discussed below, however, given the outputs will be somewhat contingent on each other, some will be discussed in relation to one another.

### Electrode Configuration Optimization

With reference to FIG. 8, the determination of electrode configuration as performed through the “optimize electrode configuration” module 150 including quantity of necessary electrodes, which can include a single electrode design or a combination of different electrode designs for a specific treatment environment (tissue/organ based on the region of interest input) will be overviewed.

The variables of interest are: (1) the electrode count and (2) the electrode position relative to the ROI object 250. These will be described in later detail after the other variables are described. The next set of variables described are those which can optionally be held constant to permit singular calculations of optimal electrode number and electrode position(s), or can be considered as co-variables to provide an electrode number and electrode position(s) that correspond to changes in the co-variable. These variables include: (1) stimulating parameters, (2) residual region of ROI remaining after a theoretical surgical intervention, (3) the desired volume of the ROI that needs an electric field magnitude and/or orientation meeting a certain threshold, (4) restricted zones of electrode entry, and (5) electrode pairing within the available contacts.

The remaining variables will be held constant within the machine learning environment for this particular application: (1) electrode design, (2) the virtual ROI object 250, and (3) the volumetric set of electrical properties of the tissue/organ within the ROI and surrounding tissue as provided by the virtual mesh model 240.

A electrode configuration solver 152 of the “optimize electrode configuration” module 150 conducts a systematic volumetric assessment of the region of interest (ROI object 250) relative to the coverage zone (total AOE region 270) of sufficient electric field treatment achieved surrounding each electrode, which can involve application of an electrode configuration machine learning model 156 and/or an electrode configuration parameter sweep model 154 to optimize the electrode count relative to the electrode positions. The volumetric assessment of the region of interest relative to the coverage zone is a fundamental calculation for determination of the electrode count necessary to achieve therapeutic treatment within the region of interest. The one or more simulation modules 192 simulate the effect of the modeled electrode objects 260 applied to the virtual mesh model 240 according to the set of treatment parameters. As discussed above, the effect of each individual contact object 262 of the modeled electrode object 260 within tissue is modeled as a respective AOE object 272 defining a volumetric region within the virtual space 200 and having one or more additional properties such as electric field intensity and direction within the volumetric region. The total AOE region 270 is descriptive of the total “coverage zone” relative to the region of interest and includes a volumetric summation of each respective AOE object 272 that meets the EFT coverage threshold of sufficient treatment. Cartesian positions of each modeled contact object 262 of the modeled electrode objects 260 are determined based on constraints of the electrode design models 191 stored within the reference library 190 relative to the volume of the ROI object 250, which can include the AOE definition model that dictates how each respective AOE object 272 should be modeled during simulation according to the current set of treatment parameters. This can involve a stepwise process of increasing electrode count while varying the positions of each modeled electrode object 260 relative to the ROI object 250 to maximize volumetric coverage of the AOE objects 272 within the ROI object 250 to obtain optimal X, Y, and Z coordinates within the 3D virtual space 200 for positioning of each modeled contact object 262, in addition to the corresponding electrode count. The outputs for electrode count and electrode positions can be iteratively re-calculated as other co-variables are optionally altered or swept across a range to identify the optimal set of EFT treatment parameters. This will result in differing electrode counts and electrode positions as optional co-variables are altered until one or more optimal electrode configurations are achieved. This optional variability is the fundamental basis for the EFT optimization system 140.

Within a treatment planning environment such as visualization environment 180, a clinician could be provided with software control over the optional variables or possibly over the variables of interest (electrode count and electrode position). The electrode configuration solver 152 could then computationally represent the outputs based on the specified variables by the electrode configuration machine learning model 156, the electrode configuration parameter sweep model 154 or both. This enables the EFT optimization system 140 to generate machine-learning provided practice-guiding outputs. Such a system could permit a practitioner to define the placement of one particular modeled electrode object 260 and receive feedback for one or more additional modeled electrode objects 260 through use of the electrode configuration machine learning model 156 fulfilling the task of populating the ROI object 250 with respect to the individual AOE objects 272 provided by each modeled electrode object 260 as they are expected to propagate through the tissue as modeled within the virtual mesh model 240. The output of the electrode configuration machine learning model 156 can be facilitated by a plurality of possible machine learning methods including supervised, unsupervised, or reinforcement learning environments, and can be structured according to examples outlined in FIG. 15.

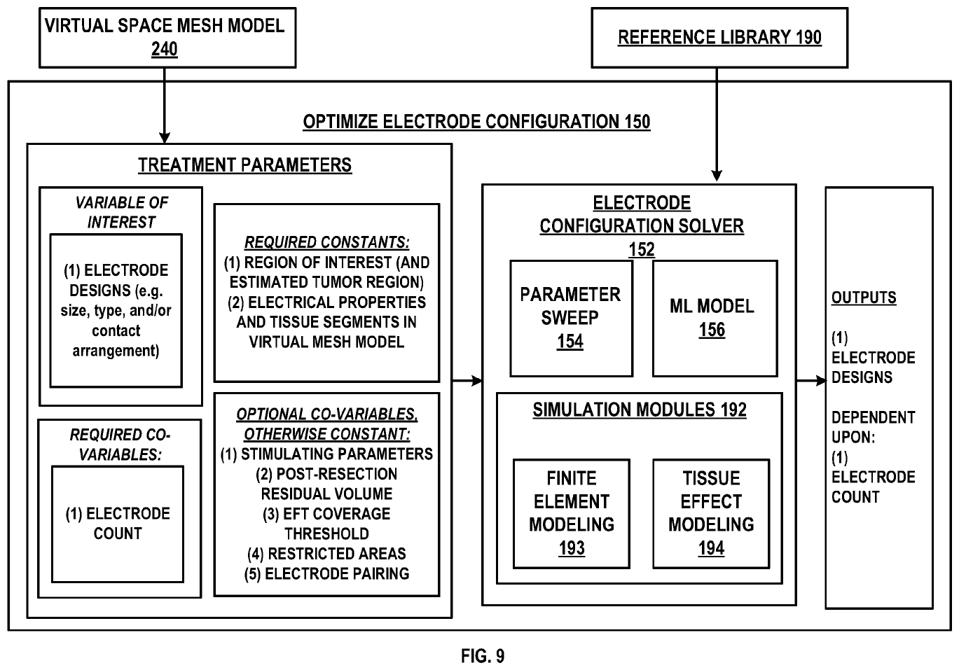

The electrode configuration solver 152 outlined above with respect to FIG. 8 used an input of fixed electrode design parameters to achieve an output of an electrode count and electrode position. However, with reference to FIG. 9, the electrode configuration solver 152 can use the variables in a different manner to output possible optimized electrode design parameters through the localization of positions of one or more contact objects 262 or groupings of contact objects 262 relative to the ROI object 250 that provide optimal electric field therapy with respect to electric field magnitude and orientation, dependent upon electrode count. The “electrode design parameters” as discussed herein describes dimensions of each modeled electrode object 260 to be applied, as well as count and positions of contact objects 262 and/or groupings of contact objects 262. For instance, with brief reference to FIG. 6A, electrode design parameters for modeled electrode object 260A can include a quantity of contact objects 262, roles of contact objects 262, and dimensional and electrical properties of the modeled electrode object 260A and associated contact objects 262 that affect the resultant AOE objects 272 for the modeled electrode object 260A. For the example of modeled electrode object 260C, the electrode design parameters can include a quantity and geometric arrangement of contact objects 262, roles of contact objects 262, and dimensional and electrical properties of the modeled electrode object 260A and associated contact objects 262 that affect the resultant AOE objects 272 for the modeled electrode object 260A. In some embodiments, alternative electrode design parameters can also be incorporated that involve a combination of one or more types of electrodes, such as the “penetrating” electrode of modeled electrode object 260A and the “surface grid” electrode of modeled object 260C, combined into one modeled electrode object with individual contacts of each respective “sub-electrode” being considered as a sub-grouping of contacts that have a pre-defined relationship with one another.

This optimization task will be conducted in a similar manner to the one described in reference to FIG. 8 for the electrode count/position, except the electrode design parameters including geometric association of each respective contact object 262 or groupings of contact objects 262 of the modeled electrode objects 260 relative to one another will be the primary variable of interest and the co-variable of electrode count will be simultaneously adjusted to determine the optimal electrode design parameters of modeled electrode objects 260 to treat the region of interest modeled by the ROI object 250. Similar to the electrode count/position application described above with reference to FIG. 8, there will be variables that can remain optionally constant, and some that will remain constant throughout the machine learning application. Optional co-variables include: (1) stimulating parameters, (2) residual region of ROI remaining after a theoretical surgical intervention, (3) the volume of the ROI achieving a threshold electric field magnitude and/or orientation, (4) restricted zones of electrode entry, and (5) electrode pairing within the available contacts. Required constants include: (1) the ROI object 250 and (2) electrical properties of the tissue/organ within the ROI and surrounding tissue as provided by the virtual mesh model 240. Electrode position will not be a variable in this part of the analysis, because positional determination would require defined electrode design parameters and therefore during this optimization task where electrode design parameters are not defined (but an output) will exclude this variable from inclusion.

The electrode configuration solver 152 conducts a systematic volumetric assessment of the region of interest relative to the coverage zone of sufficient electric field treatment achieved surrounding each electrode, which can involve application of an electrode configuration machine learning model 156 and/or an electrode configuration parameter sweep model 154 to optimize design parameters of the modeled electrode objects 260 including positions of each respective contact object 262 along the associated modeled electrode object 260 relative to the ROI object 250. The one or more simulation modules 192 simulate the effect of the modeled electrode objects 260 applied to the virtual mesh model 240 according to the set of treatment parameters as they are varied. As discussed above, the effect of each individual modeled electrode object 260 within tissue is modeled as a respective AOE object 272 defining a volumetric region within the virtual space 200 and having one or more additional properties such as electric field intensity and direction within the volumetric region. The total AOE region 270 is descriptive of the total “coverage zone” relative to the region of interest and includes a volumetric summation of each respective AOE object 272 that meets the EFT coverage threshold of sufficient treatment. In one aspect, optimized electrode configuration parameters result in a maximal EFT effect throughout the region of interest, which can be interpreted as the maximal total AOE region 270 relative to the ROI object 250. modeled electrode

### Resection Region Check

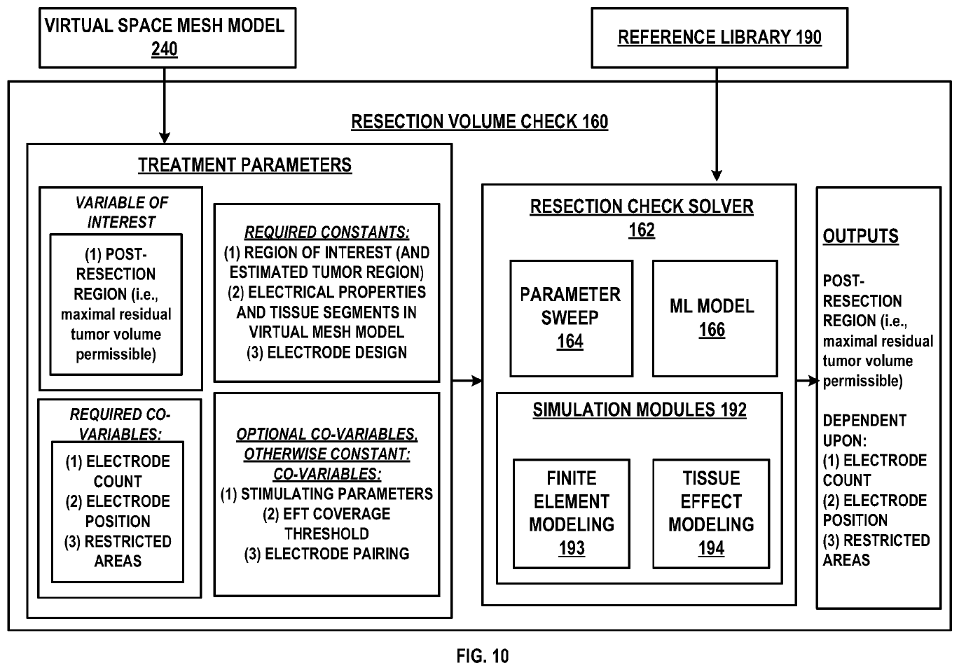

With reference to FIG. 10, the “resection region check” module 160 of the EFT optimization system 140 includes a resection check solver 162 that provides an additional clinically relevant output defining a maximal post-resection residual region of a tumor, as defined within the confines of the ROI object 250 that would enable the optimal application of EFT. To compute this analysis, the electrode design parameters of each modeled electrode object 260 are held as constants, however the quantity of modeled electrode objects 260 and position of each modeled electrode object 260 are maintained as required co-variables such that the treatment volume can be optimized across a range of electrode configurations. Importantly, given the patient-specific nature of this calculation, a co-variable that describes implant entry within the ROI object 250 and restricted areas within or around the ROI object 250 where modeled electrode objects 260 cannot pass will be included. This will allow the output of the resection check solver 162 to be clinically relevant and patient specific. The resection check solver 162 conducts a systematic volumetric assessment of the ROI object 250 relative to the total AOE region 270 descriptive of the maximum expected coverage zone of sufficient electric field treatment achieved surrounding each electrode, which can apply a resection check machine learning model 166 and/or a resection check parameter sweep model 164 to determine a maximal post-resection residual tumor region including shape, volume, and position relative to the region of interest that is permissibly allowed based on the estimated effect of the EFT. The resection check solver 162 compares the total AOE region 270 as simulated with the estimated tumor region 280 to identify a tumor coverage region where the total AOE region 270 and the estimated tumor region 280 intersect. The remaining volumetric region of the estimated tumor region 280 that is not part of the tumor coverage region and would not be sufficiently treated with present parameters can be considered for resection. The maximal post-resection residual tumor region can be defined as the tumor coverage region within the estimated tumor region 280 that is also covered by the total AOE region 270 when simulated according to the set of EFT treatment parameters that result in a maximal EFT effect throughout the region of interest. Similar to above, the total AOE region 270 descriptive of the total estimated effect of the EFT as applied to the virtual mesh model 240 through the modeled electrode objects 260 can be modeled using one or more simulation modules 192 including the finite element modeling module 193 and the tissue effect modeling module 194. Notably, the “resection region check” module 160 will include similar optional co-variables outlined in the previous discussions with reference to FIGS. 8 and 9. The output of the resection check machine learning model 166 can be facilitated by a plurality of possible machine learning methods including supervised, unsupervised, or reinforcement learning environments, and can be structured according to examples outlined in FIG. 15.

### Stimulating Parameter Optimization

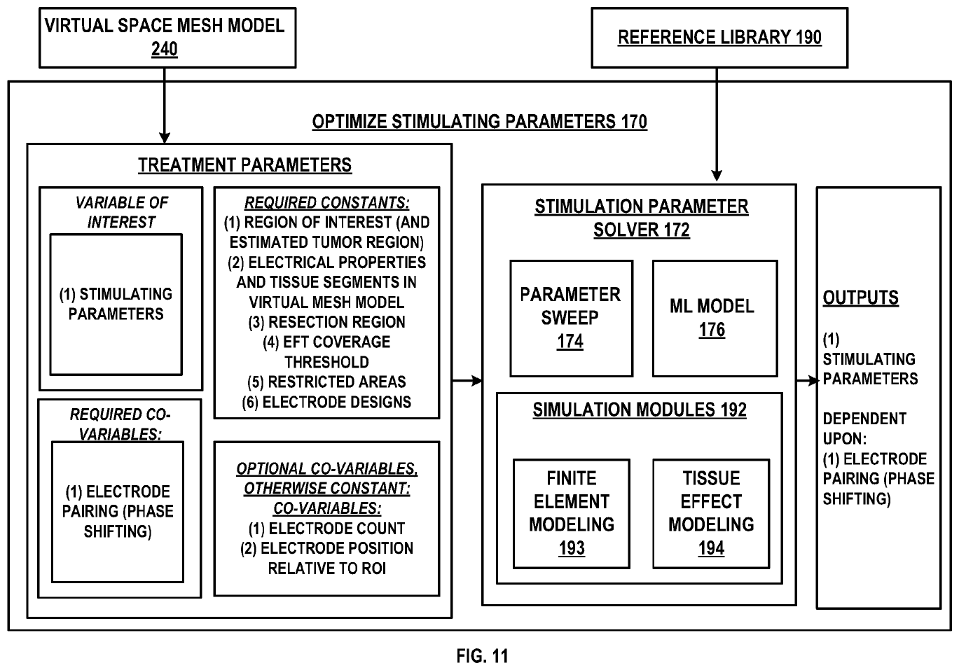

Referring to FIG. 11, the “optimize stimulating parameters” module 170 of the EFT optimization system 140 includes a stimulating parameter solver 172 that optimizes one or more stimulating parameters, such as waveform parameters, to be applied to the region of interest by modeling and optimizing the stimulating parameters and their estimated effect on tissue relative to the ROI object 250 with respect to the one or more modeled electrode objects 260. In particular, the stimulating parameter solver 172 identifies ideal stimulating parameters either through a voltage-controlled or current-controlled circuit across a complex system of electrode contacts (which are provided to the stimulating parameter solver 172 as contact objects 262 of the one or more modeled electrode objects 260). The ideal stimulating parameters can be determined based on a stimulating parameter machine learning model 176 given these parameters are dependent on the electrode count and electrode position relative to the region of interest in a patient specific manner. Given these multiple co-variables, the simulation modules 192 including the finite element modeling module 193 and the tissue effect modeling module 194 can provide case-specific quantitative support for the resulting therapeutic electric field strength and orientation that results from a given voltage or current input to an electrode system, modeled within the 3D virtual space 200 as the total AOE region 270 resultant of the one or more modeled electrode objects 260 with respect to the virtual mesh model 240 and the ROI object 250. Similar to above, the stimulating parameter solver 172 conducts a systematic volumetric assessment of the ROI object 250 relative to the coverage zone of sufficient electric field treatment achieved surrounding each electrode, which can apply the stimulating parameter machine learning model 176 and/or a stimulating parameter sweep model 174 to determine various stimulating parameters including frequency, amplitude, and waveform shape based on the estimated effect of the EFT. The output of the stimulating parameter machine learning model 176 can be facilitated by a plurality of possible machine learning methods including supervised, unsupervised, or reinforcement learning environments, and can be structured according to examples outlined in FIG. 15. In some embodiments, the one or more simulation modules 192 simulate the effect of the modeled electrode objects 260 applied to the virtual mesh model 240 according to the set of treatment parameters as they are varied. As discussed above, the effect of each individual modeled electrode objects 260 within tissue is modeled as a respective AOE object 272 defining a volumetric region within the virtual space 200 and having one or more additional properties such as electric field intensity and direction within the volumetric region. The total AOE region 270 is descriptive of the total “coverage zone” relative to the region of interest and includes a volumetric summation of each respective AOE object 272 that meets the EFT coverage threshold of sufficient treatment. In one aspect, optimized stimulating waveform parameters result in a maximal EFT effect throughout the region of interest, which can be interpreted as the maximal total AOE region 270 relative to the ROI object 250. Notably, the stimulating parameter solver 172 can also adopt an additional layer of complexity by considering phase-shifting pairs of electrodes. Within a phase-shifted pair of electrodes, the stimulating parameter solver 172 identifies two electrodes (as modeled electrode objects 260) based on geometric location within 3D virtual space 200 that are advantageously positioned to target a deficient zone of the ROI object 250 in between the two electrodes. Phase shifting of a sinusoid stimulatory waveform allows for an enhanced perceived electric field magnitude between two electrodes despite a consistently applied input voltage when a 180-degree phase shifting combination is adopted. The pairing of electrodes also permits the control of time-lapsed electric field orientation within the interval region between the electrodes. The stimulating parameter solver 172 could also compute a favorable sequence of pairing strategies between the available implanted electrodes. This would allow for each electrode to function independent of other electrodes in complex pairing schemes that optimize the delivered electric field therapy within the region of interest. Notably, to accomplish this analysis more variables are required to remain constant, so this analysis would ideally be conducted after the treatment system has already been implanted or has been already defined (held constant). Therefore, the electrode configuration parameters including electrode position, electrode design and electrode count are likely to remain constant during this analysis, however, a detailed analysis could hypothetically be undertaken where electrode position, electrode design and electrode count are also added in as co-variables.

### Additional Machine Learning Applications

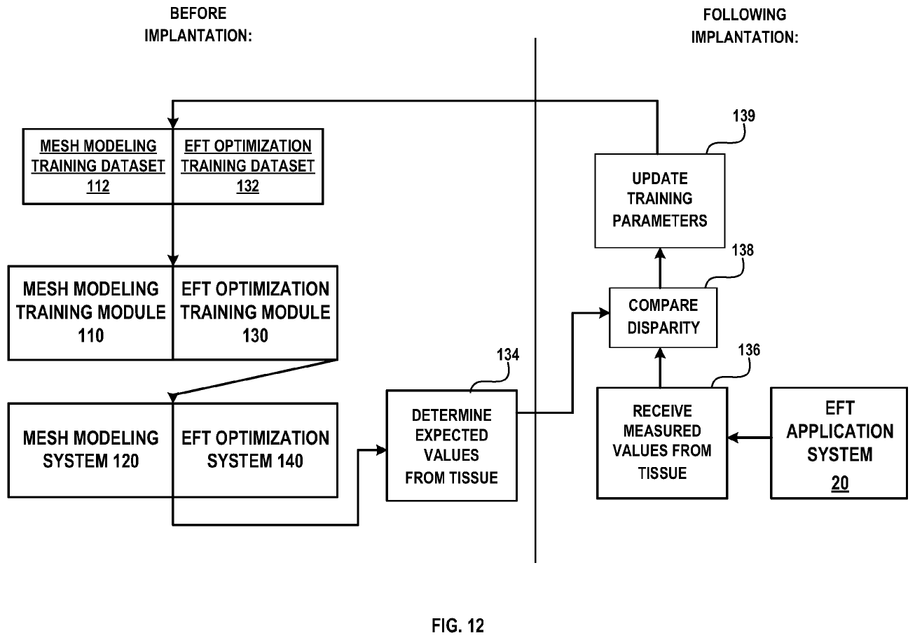

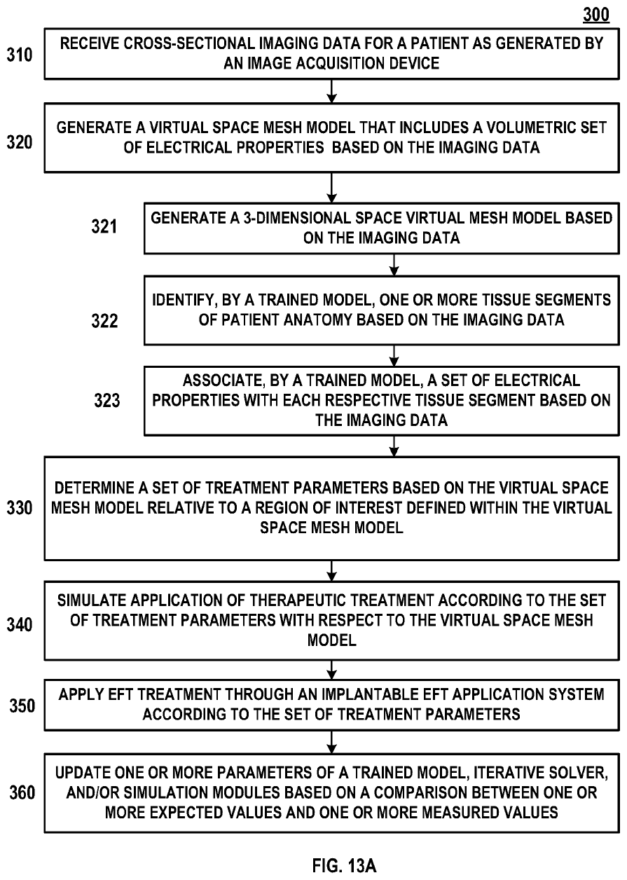

Referring to FIG. 12, a strategy for iteratively improving the EFT planning system 100 overtime is provided. As shown, the mesh modeling system 120 can be trained on one or more tissue segmentation and electrical property estimation tasks by the mesh modeling training module 110 based on mesh modeling training dataset 112, which can include training case imaging data and associated hand-segmented tissue regions and electrical properties. EFT optimization system 140 can be trained on one or more optimization tasks by the EFT optimization training module 130 based on EFT optimization training data 132, which can include virtual space mesh models and associated sets of EFT treatment parameters for “master” cases. As discussed, while the EFT optimization system 140 optimizes the set of EFT treatment parameters, the EFT optimization system 140 determines expected values within the tissue resultant of the applied set of EFT treatment parameters, such as electric field strength and direction values, range of coverage (total AOE), voltages and/or current values at specific locations within the tissue (block 134), and the calculations can also be dependent upon the virtual mesh model representative of the tissue if the virtual mesh model is provided by the mesh modeling system 120. Determining the expected values is achieved through simulation using the simulation modules 192 including the finite element modeling module 193 and the tissue effect modeling module 194. This information can be leveraged following implantation of the implantable EFT application system 20 according to the set of EFT treatment parameters found using the EFT optimization system 140. The implantable EFT application system 20 can be configured to receive or otherwise determine real-world measured values from the tissue (block 136), which can be compared directly to the expected values modeled by the EFT optimization system 140 (block 138) and used to update one or more training parameters of the EFT optimization system 140 (block 139), including one or more simulation or modeling parameters of the finite element modeling module 193 and the tissue effect modeling module 194, and optionally including the electrode configuration machine learning model 156, resection check machine learning model 166 and stimulating parameter machine learning model 176.

As such, the establishment of an anatomically based model for the virtual finite element analysis permits the EFT planning system 100 to “learn” from previous patient's modulatory data (i.e. voltage measurements within given tissue types, or impedance detection) to permit a correction of dielectric property assignments either for that patient or for other patients in the pre-implantation or post-implantation environment.

Voltage sampling acquired from the implantable EFT application system 20 an example of real-world data that provides an input for algorithmic modification. When running the simulation by the simulation modules 192, the EFT planning system 100 should predict a precise voltage experienced by a measurement electrode within a cartesian space housed within the region of interest (or adjacent). If post-implantation real-world voltage sampling measurements obtained through the implantable EFT application system 20 convey a different reality than that predicted by the EFT planning system 100, this correction can be applied to the EFT optimization system 140 through modification of the electrical properties being utilized to permit the finite element modeling or through a corrective factor being used as a multiplier for modeling provided outputs. Similarly, errors within the mesh modeling system 120 can be applied based on the perceived real-world value for voltage acquired by the implantable EFT application system 20. The virtual objects (and therefore the regions associated electrical properties) could be adjusted based on the voltage sampling results to accommodate for the perceived mismatch between the two, permitted a potentially broader application of these refinements to other patient-specific cases, if such a trend is noted to be consistently observed. Over time, a robust collection of real-world datapoints for voltage, as well as other datapoints of interest (such as temperature acquisition, impedance mapping through test stimuli, etc.) permit refinement of the EFT planning system 100.

### Process Flows

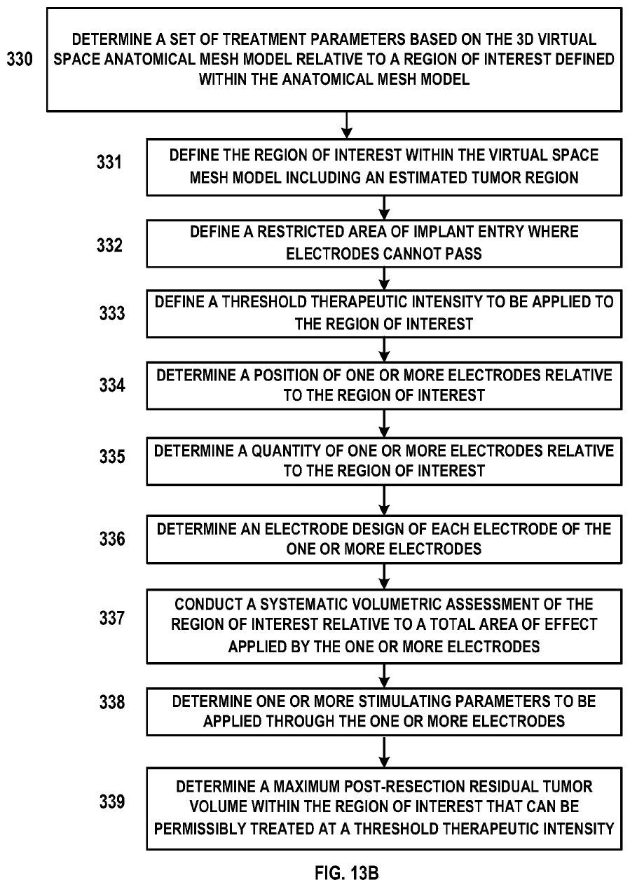

Referring to FIGS. 13A and 13B, a simplified process flow 300 is provided that enables planning and optimization of EFT treatment by the EFT planning system 100 of FIGS. 1A-12 and in conjunction with the implantable EFT application system 20 of FIGS. 1A and 12.

At step 310 of process flow 300, the EFT planning system 100 receives one or more cross-sectional image sets for a patient as generated by the image acquisition device 10. At step 320, the EFT planning system 100 generates a virtual space mesh model that includes a volumetric set of electrical properties based on the imaging data. Step 320 can include sub-steps outlined in FIG. 13A such as step 321 in which the EFT planning system 100 generates a 3D space virtual mesh model based on the imaging data. At step 322, the EFT planning system 100 identifies, by a trained model, one or more tissue segments of patient anatomy based on the imaging data. At step 323, the EFT planning system 100 associates, by a trained model, a volumetric set of electrical properties with each respective tissue segment based on the imaging data.

At step 330 of process flow 300, the EFT planning system 100 determines a set of treatment parameters based on the virtual space mesh model relative to a region of interest defined within the virtual space mesh model. Step 330 can involve sub-steps outlined in FIG. 13B such as step 331 in which the EFT planning system 100 defines the region of interest including the estimated tumor region within the virtual space mesh model. At step 332, the EFT planning system 100 defines a restricted area of implant entry within the virtual space mesh model where electrodes cannot pass. At step 333, the EFT planning system 100 defines a threshold therapeutic intensity to be applied to the region of interest. At step 334, the EFT planning system 100 determines a position of one or more electrodes relative to the region of interest. At step 335, the EFT planning system 100 determines a quantity of one or more electrodes relative to the region of interest. At step 336, the EFT planning system 100 determines an electrode design of each electrode of the one or more electrodes. At step 337, the EFT planning system 100 conducts a systematic volumetric assessment of the region of interest relative to the total area of effect applied by the one or more electrodes based on the electrode configuration parameters determined in steps 334-336 to determine a correctness of the result of each step. It should be noted that step 337 can be performed multiple times as treatment parameters are changed and identified and can also be performed between each step 334-336. At step 338, the EFT planning system 100 determines one or more stimulating parameters to be applied through the one or more electrodes. Step 337 can be repeated after step 338 to determine a correctness of the result of step 338. At step 339, the EFT planning system 100 determines a maximum post-resection residual tumor region within the region of interest that can be permissibly treated at a threshold therapeutic intensity.

At step 340 of process flow 300 shown in FIG. 13A, the EFT planning system 100 simulates application of therapeutic treatment according to the set of treatment parameters with respect to the virtual space mesh model. It should be noted that step 340 can be performed many times and can also be performed between any of steps 330-339 as a correction check and/or can be part of an iterative solving strategy that “sweeps” EFT treatment parameters through simulation to identify the optimal set of EFT treatment parameters.

At step 350 of process flow 300, the implantable EFT application system 20 applies EFT treatment according to the set of EFT treatment parameters found by the EFT planning system 100. At step 360, the EFT planning system 100 updates one or more of its parameters, including those of a trained machine-learning model or iterative solver and those of one or more simulating modules of the EFT planning system 100 based on a comparison between one or more expected values and one or more measured values obtained from the implantable EFT application system 20.

### Computer-Implemented System

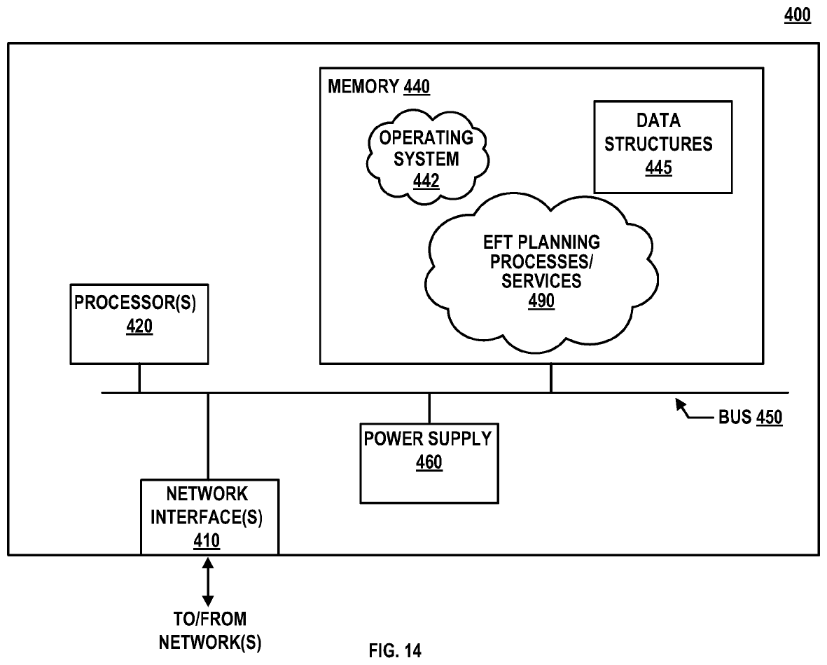

FIG. 14 is a schematic block diagram of an example device 400 that may be used with one or more embodiments described herein, e.g., as a component of system 100 shown in FIGS. 1A and 1B.

Device 400 comprises one or more network interfaces 410 (e.g., wired, wireless, PLC, etc.), at least one processor 420, and a memory 440 interconnected by a system bus 450, as well as a power supply 460 (e.g., battery, plug-in, etc.).

Network interface(s) 410 include the mechanical, electrical, and signaling circuitry for communicating data over the communication links coupled to a communication network. Network interfaces 410 are configured to transmit and/or receive data using a variety of different communication protocols. As illustrated, the box representing network interfaces 410 is shown for simplicity, and it is appreciated that such interfaces may represent different types of network connections such as wireless and wired (physical) connections. Network interfaces 410 are shown separately from power supply 460, however it is appreciated that the interfaces that support PLC protocols may communicate through power supply 460 and/or may be an integral component coupled to power supply 460.

Memory 440 includes a plurality of storage locations that are addressable by processor 420 and network interfaces 410 for storing software programs and data structures associated with the embodiments described herein. In some embodiments, device 400 may have limited memory or no memory (e.g., no memory for storage other than for programs/processes operating on the device and associated caches).

Processor 420 comprises hardware elements or logic adapted to execute the software programs (e.g., instructions) and manipulate data structures 445. An operating system 442, portions of which are typically resident in memory 440 and executed by the processor, functionally organizes device 400 by, inter alia, invoking operations in support of software processes and/or services executing on the device. These software processes and/or services may include EFT planning processes/services 490 that implement aspects of the EFT planning system 100 described herein. Note that while EFT planning processes/services 490 is illustrated in centralized memory 440, alternative embodiments provide for the process to be operated within the network interfaces 410, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the EFT planning processes/services 490 is shown as a standalone process, those skilled in the art will appreciate that this process may be executed as a routine or module within other processes.

### Machine Learning Models

FIG. 15 is a schematic block diagram of an example neural network architecture 500 that may be used with one or more embodiments described herein, e.g., as a component of system 100 shown in FIGS. 1A and 1B, and particularly as a component of the tissue segmentation module 124 and the electrical property estimation module 126 of mesh modeling system 120 (FIG. 2) and/or as a component of the electrode configuration machine learning model 156, resection check machine learning model 166, and stimulating parameter machine learning model 176 of EFT optimization system 140 (FIGS. 8-11).

Architecture 500 includes a neural network 510 defined by an example neural network description 501 in an engine model (neural controller) 530. The neural network 510 can represent a neural network implementation of a tissue segmentation engine, electrical property annotation engine, and/or treatment parameter optimization engine for segmenting tissue types within cross-sectional patient imaging, annotating segmented tissue types, and optimizing treatment parameters for application of EFT treatment. The neural network description 501 can include a full specification of the neural network 510, including the neural network architecture 500. For example, the neural network description 501 can include a description or specification of the architecture 500 of the neural network 510 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

The neural network 510 reflects the architecture 500 defined in the neural network description 501. In a first example corresponding to mesh modeling system 120, the neural network 510 includes an input layer 502, which includes input data, such as a set of cross-sectional images of patient anatomy as acquired from image acquisition device 10 (FIG. 1A), with individual “slices” or sections within individual slices corresponding to one or more nodes 508. In one illustrative example, the input layer 502 can include data representing a portion of input media data such as a patch of data or pixels (e.g., a 128×128 patch of data) in a cross-sectional image corresponding to the input media data (e.g., that of the set of cross-sectional images of patient anatomy).

In another example corresponding to EFT optimization system 140, the neural network 510 includes an input layer 502, which includes input data, such as a 3D virtual space mesh model 240 (FIGS. 3D and 6) representative of patient anatomy, with sub-volumes and objects such as an ROI object 250 and one or more modeled electrode objects 260 with associated design parameters corresponding to one or more nodes 508. In one illustrative example, the input layer 502 can include data representing a portion of input media data such as a patch of data or voxels (e.g., a 128×128×128 patch of data) in a voxel image corresponding to the input media data (e.g., that of the 3D virtual space mesh model 240).

The neural network 510 includes hidden layers 504A through 504N (collectively “504” hereinafter). The hidden layers 504 can include n number of hidden layers, where n is an integer greater than or equal to one. The number of hidden layers can include as many layers as needed for a desired processing outcome and/or rendering intent. The neural network 510 further includes an output layer 506 that provides an output (e.g., tissue segments, electrical property annotations, suggested treatment parameters) resulting from the processing performed by the hidden layers 504. In a first illustrative example corresponding to the mesh modeling system 120, the output layer 506 can provide tissue segments and a volumetric set of electrical properties of patient anatomy as identifiable within the set of cross-sectional images of patient anatomy provided to the input layer 502. In another illustrative example corresponding to the EFT optimization system 140, the output layer 506 can provide suggested treatment parameters including electrode configuration parameters such as modeled electrode design parameters, modeled electrode count, positions of modeled electrodes relative to ROI, and also including stimulating parameters such as those corresponding to a waveform to be applied by each respective electrode based on the 3D virtual space mesh model of patient anatomy provided to the input layer 502.

The neural network 510 in this example is a multi-layer neural network of interconnected nodes. Each node can represent a piece of information. Information associated with the nodes is shared among the different layers and each layer retains information as information is processed. In some cases, the neural network 510 can include a feed-forward neural network, in which case there are no feedback connections where outputs of the neural network are fed back into itself. In other cases, the neural network 510 can include a recurrent neural network, which can have loops that allow information to be carried across nodes while reading in input.

Information can be exchanged between nodes through node-to-node interconnections between the various layers. Nodes of the input layer 502 can activate a set of nodes in the first hidden layer 504A. For example, as shown, each of the input nodes of the input layer 502 is connected to each of the nodes of the first hidden layer 504A. The nodes of the hidden layer 504A can transform the information of each input node by applying activation functions to the information. The information derived from the transformation can then be passed to and can activate the nodes of the next hidden layer (e.g., 504B), which can perform their own designated functions. Example functions include convolutional, up-sampling, data transformation, pooling, and/or any other suitable functions. The output of the hidden layer (e.g., 504B) can then activate nodes of the next hidden layer (e.g., 504N), and so on. The output of the last hidden layer can activate one or more nodes of the output layer 506, at which point an output is provided. In some cases, while nodes (e.g., nodes 508A, 508B, 508C) in the neural network 510 are shown as having multiple output lines, a node has a single output and all lines shown as being output from a node represent the same output value.

In some cases, each node or interconnection between nodes can have a weight that is a set of parameters derived from training the neural network 510. For example, an interconnection between nodes can represent a piece of information learned about the interconnected nodes. The interconnection can have a numeric weight that can be tuned (e.g., based on a training dataset), allowing the neural network 510 to be adaptive to inputs and able to learn as more data is processed.

The neural network 510 can be pre-trained to process the features from the data in the input layer 502 using the different hidden layers 504 in order to provide the output through the output layer 506. In an example corresponding to mesh modeling system 120 in which the neural network 510 is used to identify tissue segments and/or a volumetric set of estimated electrical properties based on the set of cross-sectional imaging representative of patient anatomy, the neural network 510 can be trained using training data that includes example cross-sectional images and hand-segmented tissue data and/or annotated electrical properties for individual cross-sectional image “slices” from a training dataset (i.e. mesh modeling training data 112 of FIGS. 1B and 2). In another example corresponding to EFT optimization system 140 in which the neural network 510 is used to provide a set of treatment parameters based on the 3D virtual space mesh model 240 representative of patient anatomy, the neural network 510 can be trained using training data that includes example 3D virtual space mesh models and corresponding treatment parameters from hand-selected “master” training cases provided by “master” surgeons (i.e. EFT optimization training data 132 of FIG. 1B). For instance, training data can be input into the neural network 510, which can be processed by the neural network 510 to generate outputs which can be used to tune one or more aspects of the neural network 510, such as weights, biases, etc.

In some cases, the neural network 510 can adjust weights of nodes using a training process called backpropagation. Backpropagation can include a forward pass, a loss function, a backward pass, and a weight update. The forward pass, loss function, backward pass, and parameter update is performed for one training iteration. The process can be repeated for a certain number of iterations for each set of training media data until the weights of the layers are accurately tuned.

For a first training iteration for the neural network 510, the output can include values that do not give preference to any particular class due to the weights being randomly selected at initialization. For example, if the output is a vector with probabilities that the object includes different product(s) and/or different users, the probability value for each of the different product and/or user may be equal or at least very similar (e.g., for ten possible products or users, each class may have a probability value of 0.1). With the initial weights, the neural network 510 is unable to determine low level features and thus cannot make an accurate determination of what the classification of the object might be. A loss function can be used to analyze errors in the output. Any suitable loss function definition can be used.

The loss (or error) can be high for the first training dataset (e.g., images) since the actual values will be different than the predicted output. The goal of training is to minimize the amount of loss so that the predicted output comports with a target or ideal output. The neural network 510 can perform a backward pass by determining which inputs (weights) most contributed to the loss of the neural network 510, and can adjust the weights so that the loss decreases and is eventually minimized.

A derivative of the loss with respect to the weights can be computed to determine the weights that contributed most to the loss of the neural network 510. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 510 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. In other examples, the neural network 510 can represent any other neural or deep learning network, such as an autoencoder, a deep belief nets (DBNs), and recurrent neural networks (RNNs), etc.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a memory, the memory including instructions, which, when executed, cause the processor to:
generate a virtual space mesh model representative of an anatomical structure based on a set of cross-sectional imaging data, the virtual space mesh model including a volumetric set of electrical properties; and
determine, based on the virtual space mesh model relative to a region of interest defined within the virtual space mesh model, a set of electric field therapy treatment parameters to be applied to the anatomical structure by an implantable electric field therapy application system such that a resultant area of effect of the implantable electric field therapy application system reaches a sufficient coverage threshold across the region of interest, the set of electric field therapy treatment parameters including at least one of:
one or more electrode configuration parameters of the implantable electric field therapy application system;
one or more stimulating parameters descriptive of a stimulating waveform to be applied through the implantable electric field therapy application system; and
a maximal permissible post-resection residual region of a tumor within the region of interest.

2. The system of claim 1, wherein the memory further includes instructions, which, when executed, cause the processor to:
identify one or more tissue segments of patient anatomy based on the set of cross-sectional imaging data, and
associate one or more electrical properties of the volumetric set of electrical properties with each respective tissue segment of the one or more tissue segments.

3. The system of claim 2, wherein the memory includes one or more machine learning models operable to identify the one or more tissue segments within the set of cross-sectional imaging data and associate one or more electrical properties with each respective tissue segment of the one or more tissue segments within a set of patient imaging data.

4. The system of claim 1, wherein the set of cross-sectional imaging data includes a plurality of cross-sectional anatomical images of an anatomical structure obtained through one or more magnetic resonance imaging methods.

5. The system of claim 1, wherein the memory further includes instructions, which, when executed, cause the processor to:
conduct a systematic volumetric assessment of the region of interest defined within the virtual space mesh model relative to an expected coverage zone applied by one or more electrodes of the implantable electric field therapy application system with respect to the set of electric field therapy treatment parameters and the virtual space mesh model.

6. The system of claim 5, wherein the memory further includes instructions, which, when executed, cause the processor to:
simulate application of electric field therapy to the virtual space mesh model according to the set of electric field therapy treatment parameters by one or more modeled electrode objects representative of one or more electrodes of the implantable electric field therapy application system.

7. The system of claim 6, wherein the memory further includes instructions, which, when executed, cause the processor to:
sweep one or more electric field therapy treatment parameters of the set of electric field therapy treatment parameters across a range during iterative simulation of the application of electric field therapy to the virtual space mesh model; and
determine one or more electric field therapy treatment parameters of the set of electric field therapy treatment parameters that result in an area of effect of the one or more electrodes reaching a sufficient coverage threshold across the region of interest.

8. The system of claim 5, wherein the memory further includes instructions, which, when executed, cause the processor to:
determine, by a machine learning model, one or more electric field therapy treatment parameters of the set of electric field therapy treatment parameters based on a similarity of the virtual space mesh model to one or more master cases.

9. The system of claim 1, wherein the one or more electrode configuration parameters includes at least one of:
a quantity of one or more electrodes of the implantable electric field therapy application system to be implanted within tissue;
one or more electrode design parameters of the one or more electrodes of the implantable electric field therapy application system; and
a position of each electrode of the one or more electrodes of the implantable electric field therapy application system relative to the region of interest.

10. The system of claim 1, wherein the memory further includes instructions, which, when executed, cause the processor to:
update one or more parameters of the system based on a comparison between one or more expected values and one or more measured values following application of electric field therapy to the anatomical structure through the implantable electric field therapy application system.

11. A method, comprising:
generating, by a processor in communication with a memory, a virtual space mesh model representative of an anatomical structure based on a set of cross-sectional imaging data, the virtual space mesh model including a volumetric set of electrical properties; and
determining, by the processor and based on the virtual space mesh model relative to a region of interest defined within the virtual space mesh model, a set of electric field therapy treatment parameters to be applied to the anatomical structure by an implantable electric field therapy application system such that a resultant area of effect of the implantable electric field therapy application system reaches a sufficient coverage threshold across the region of interest, the set of electric field therapy treatment parameters including at least one of:
one or more electrode configuration parameters of the implantable electric field therapy application system;
one or more stimulating parameters descriptive of a waveform to be applied through the implantable electric field therapy application system; and
a maximal permissible post-resection residual region of a tumor within the region of interest.

12. The method of claim 11, further comprising:
identifying, by a first machine learning model in association with the processor, one or more tissue segments of patient anatomy based on the set of cross-sectional imaging data; and
associating, by a second machine learning model in association with the processor, one or more electrical properties of the volumetric set of electrical properties with each respective tissue segment of the one or more tissue segments.

13. The method of claim 12, further comprising:
training, by a processor, the first machine learning model to identify the one or more tissue segments within the set of cross-sectional imaging data using a set of training data that demonstrates tissue segmentation for a plurality of cross-sectional images across a plurality of training cases; and
training, by a processor, the second machine learning model to associate set of electrical properties with each respective tissue segment of the one or more tissue segments within the set of cross-sectional imaging data using a set of training data that demonstrates estimation of a set of electrical properties for a plurality of cross-sectional images across a plurality of training cases.

14. The method of claim 11, further comprising:
obtaining the set of cross-sectional imaging data for a patient using an image acquisition device, wherein the image acquisition device is a magnetic resonance imaging device.

15. The method of claim 11, further comprising:
conducting, by the processor, a systematic volumetric assessment of the region of interest defined within the virtual space mesh model relative to an expected coverage zone applied by one or more electrodes of the implantable electric field therapy application system with respect to the set of electric field therapy treatment parameters and the virtual space mesh model.

16. The method of claim 15, further comprising:
simulating, by the processor, application of electric field therapy to the virtual space mesh model according to the set of electric field therapy treatment parameters by one or more modeled electrode objects representative of the one or more electrodes of the implantable electric field therapy application system.

17. The method of claim 16, further comprising:
sweeping, by the processor, one or more electric field therapy treatment parameters of the set of electric field therapy treatment parameters across a range during iterative simulation of the application of electric field therapy to the virtual space mesh model; and
determining, by the processor, one or more electric field therapy treatment parameters of the set of electric field therapy treatment parameters that result in an area of effect of the one or more electrodes of the implantable electric field therapy application system reaching a sufficient coverage threshold across the region of interest.

18. The method of claim 15, further comprising:
determining, by a third machine learning model, one or more electric field therapy treatment parameters of the set of electric field therapy treatment parameters based on a similarity of the virtual space mesh model to one or more master cases.

19. A system, comprising:
a processor in communication with a memory, the memory including instructions, which, when executed, cause the processor to:
receive a virtual space mesh model representative of an anatomical structure based on a set of cross-sectional imaging data, the virtual space mesh model including a set of electrical properties associated with each voxel of a plurality of voxels present within the virtual space mesh model; and
determine, based on the virtual space mesh model relative to a region of interest defined within the virtual space mesh model, a set of electric field therapy treatment parameters to be applied to the anatomical structure by an implantable electric field therapy such that a resultant area of effect of the implantable electric field therapy application system reaches a sufficient coverage threshold across the region of interest, the set of electric field therapy treatment parameters including at least one of:
one or more electrode configuration parameters of the implantable electric field therapy application system;
one or more stimulating parameters descriptive of a stimulating waveform to be applied through the implantable electric field therapy application system; and
a maximal permissible post-resection residual region of a tumor within the region of interest.

20. The system of claim 19, wherein the memory further includes instructions, which, when executed, cause the processor to:
conduct a systematic volumetric assessment of the region of interest defined within the virtual space mesh model relative to an expected coverage zone applied by one or more electrodes of the implantable electric field therapy application system with respect to the set of electric field therapy treatment parameters and the virtual space mesh model.

21. The system of claim 20, wherein the memory further includes instructions, which, when executed, cause the processor to:
simulate application of electric field therapy to the virtual space mesh model according to the set of electric field therapy treatment parameters by one or more modeled electrode objects representative of the one or more electrodes of the implantable electric field therapy application system.

22. The system of claim 21, wherein the memory further includes instructions, which, when executed, cause the processor to:
sweep one or more electric field therapy treatment parameters of the set of electric field therapy treatment parameters across a range during iterative simulation of the application of electric field therapy to the virtual space mesh model; and
determine one or more electric field therapy treatment parameters of the set of electric field therapy treatment parameters that result in an area of effect of the one or more electrodes reaching a sufficient coverage threshold across the region of interest.

23. The system of claim 20, wherein the memory further includes instructions, which, when executed, cause the processor to:
determine, by a machine learning model, one or more electric field therapy treatment parameters of the set of electric field therapy treatment parameters based on a similarity of the virtual space mesh model to one or more master cases.

24. The system of claim 19, wherein the one or more electrode configuration parameters includes at least one of:
a quantity of one or more electrodes of the implantable electric field therapy application system to be implanted within tissue;
one or more electrode design parameters of the one or more electrodes of the implantable electric field therapy application system; and
a position of each electrode of the one or more electrodes of the implantable electric field therapy application system relative to the region of interest.
